(19) United States
(12) Patent Application Publication
(10) Pub. No.: US 2024/0294605 A1
(43) Pub. Date: Sep. 5, 2024

(54) A CROSS-HYBRID FC-FUSION POLYPEPTIDE TARGETING PD-L1 AND METHODS AND USES RELATED THERETO

(71) Applicant: HELSINGIN YLIOPISTO, Helsingin Yliopisto (FI)

(72) Inventors: Vincenzo CERULLO, Helsingin Yliopisto (FI); Firas HAMDAN, Helsingin Yliopisto (FI); Erkko YLÖSMÄKI, Helsingin Yliopisto (FI)

(21) Appl. No.: 18/275,753

(22) PCT Filed: Feb. 4, 2022
(86) PCT No.: PCT/FI2022/050070

§ 371 Date: Aug. 3, 2023

(30) Foreign Application Priority Data
Feb. 4, 2021 (FI) 20215115

(51) Int. Cl. C07K 14/705; A61K 38/00; A61P 35/00; C12N 15/86

(52) U.S. Cl. CPC C07K 14/70596; A61P 35/00; C12N 15/86; A61K 38/00; C07K 2319/30

(57) ABSTRACT

The present invention relates to the fields of life sciences and medicine. Specifically, the invention relates to a cross-hybrid Fc-fusion polypeptide targeting PD-L1, a polynucleotide encoding the Fc-fusion polypeptide, and a vector comprising a polynucleotide encoding the Fc-fusion polypeptide. Also, the present invention relates to a pharmaceutical composition comprising the Fc-fusion polypeptide, polynucleotide or vector of the present invention. Still, the present invention relates to a method of treating a cancer in a subject and to the Fc-fusion polypeptide, polynucleotide, vector or pharmaceutical composition for use in treatment of a cancer. Still furthermore, the present invention relates to a method of preparing the cross-hybrid Fc-fusion polypeptide of the present invention and a method of preparing the vector of the present invention.

### FIELD OF THE INVENTION

The present invention relates to the fields of life sciences and medicine. Specifically, the invention relates to a cross-hybrid Fc-fusion polypeptide targeting PD-L1, a polynucleotide encoding the Fc-fusion polypeptide, and a vector comprising a polynucleotide encoding the Fc-fusion polypeptide. Also, the present invention relates to a pharmaceutical composition comprising the Fc-fusion polypeptide, polynucleotide or vector of the present invention. Still, the present invention relates to a method of treating a cancer in a subject and to the Fc-fusion polypeptide, polynucleotide, vector or pharmaceutical composition for use in treatment of a cancer. Still furthermore, the present invention relates to a method of preparing the cross-hybrid Fc-fusion polypeptide of the present invention and a method of preparing the vector of the present invention.

### BACKGROUND OF THE INVENTION

Immune checkpoint inhibitor (ICI) therapies have been established as a potent treatment option for a plethora of tumor types and have significantly expanded the therapeutic armamentarium in oncology. Such agents target immune inhibitory receptors and interrupt co-inhibitory signaling pathways, abrogating their immunosuppressive function and consequently revitalizing anti-tumor immune response. The consequent restoration of immune-mediated elimination of tumor cells leads to long-term, sustained tumor responses. For example, the introduction of ipilimumab, an antibody against the inhibitory immune checkpoint CTLA-4, has doubled the 10-year survival for metastatic melanoma (Hodi, F. S. et al. 2010, N. Engl. J. Med. 363, 711-723; Tsao, H. et al. 2004, New England Journal of Medicine vol. 351 998-1012). Moreover, higher response and lower side effect rates have been achieved with antibodies against other inhibitory checkpoints, such as PD-1/PD-L1 inhibitors, resulting in their approval as first line treatments for a growing list of malignancies (Zhang, B. et al. 2020, BMC Cancer 20.1, 1-12).

Nevertheless, accumulating evidence has shown that checkpoint inhibitors can only benefit a fraction of patients. For instance, approximately half of the patients with metastatic melanoma do not respond to ICI therapy (Shields, B. D. et al. 2017, Sci. Rep. 7, 1-12). Intrinsic resistance to PD-1 antibodies is not uncommon since up to 60% of patients bearing some cancer types were proven to be resistant (Johnson, D. B. et al. 2015, Therapeutic Advances in Medical Oncology vol. 7, 97-106). Despite encouraging initial treatment response, acquired resistance to checkpoint inhibitors (O'Donnell, J. S. et al. 2016, Genome Med. 8, 1-3) has also been reported and severe immune-related adverse effects (irAEs) are noticed in some patients undergoing ICI therapy (Feng, Y. et al. 2013, Clin. Cancer Res. 19, 3977-3986).

All clinically approved ICIs are antibodies that primarily act as antagonizing agents with their main mechanism of action being the re-constitution of a T-cell response by disrupting an immunosuppressive axis (Pardoll, D. M. 2012, Nature Reviews Cancer vol. 12, 252-264). Nevertheless, ICIs are either limited or entirely not able to elicit crucial effector mechanisms (Chen, X. et al. 2019, Frontiers in Immunology vol. 10) such as complement-dependent cytotoxicity (CDC) or antibody-dependent cell cytotoxicity/phagocytosis (ADCC/ADCP) which are pertinent to an antibody.

Despite the success of some immune checkpoint inhibitors in the clinic, only a fraction of patients benefits from these therapies. Therefore, there is a need for more effective, safe and specific therapeutics against cancer.

### BRIEF DESCRIPTION OF THE INVENTION

The objects of the present invention, namely effective, safe and/or specific therapeutics, are achieved by utilizing a novel enhanced ICI against PD-L1 (Programmed death-ligand 1). Indeed, it has now been found that by combining a specific Fc-polypeptide and a region of PD-1 (Programmed cell death protein 1), an effective therapeutic agent (one molecule) and synergistic therapeutic effects can be obtained.

The therapeutic Fc-fusion polypeptide makes it possible e.g. to achieve tumor clearance and activate ADCC and CDC. Indeed, the cross-isotype Fc region gives the ICI the ability to elicit effector mechanisms of two different Ig isotypes in various tumor cell lines, and the subsequent activation of multiple effector mechanisms further enhance tumor killing. It is the specific design of the Fc-fusion polypeptide of the present invention which enables surprising effects on cancer cells and patient material and furthermore improved cancer treatment efficacy.

The concept of the present invention of increasing PD-L1 ICI efficacy via enhancing Fc-effector mechanisms enables excellent tumor killing and depletion of immunosuppressive populations. The present disclosure demonstrates that the simultaneous engagement of Fc-α and Fc-γ with the Fc-fusion peptides of the present invention work in synergy leading to unexpectedly enhanced tumor killing. In conclusion, the cross-hybrid Fc-fusion polypeptides of the present invention reveal that activating multiple immune effector populations increases tumor cytotoxicity leading to improved clinical outcomes.

More specifically, the inventors of the present disclosure were able to design a cross-hybrid Fc-fusion polypeptide targeting PD-L1 which polypeptide is capable of eliciting effector mechanisms of an IgG1 and IgA consequently activating polymorphonuclear leukocytes (PMNs), a population neglected by IgG1, in order to combine multiple effector mechanisms. Indeed, the inventors were able to produce a chimeric IgG-IgA (IgGA) Fc linked to a peptide region of PD1, wherein the fusion polypeptide is capable of binding to PD-L1 and activating multiple immune components enhancing tumor cytotoxicity, e.g. when compared to FDA-approved immune checkpoint inhibitors, in various cancer cell lines and carcinoma patient derived organoids. The Fc-fusion polypeptides of the present invention are not only able to activate peripheral blood mononuclear cells (PBMCs), usually activated by IgG1 antibodies, but also engage a neglected but important population, PMNs. This co-engagement of both populations was shown to work in synergy augmenting tumor killing in PD-L1 expressing cell lines and patient-derived cancer organoids.

One or more polynucleotides encoding the Fc-fusion peptides of the present invention can be included in and optionally expressed from e.g. viral vectors. For example, in order to prevent toxicity an oncolytic adenoviral vector whose replication is restricted to a tumor can be used for delivering said one or more polynucleotides encoding the Fc-fusion peptides to cells.

The present invention provides specific tools and methods for specific and stunningly effective treatment of a cancer.

Specifically, the present invention relates to a cross-hybrid Fc-fusion polypeptide targeting or against PD-L1, wherein the Fc-fusion polypeptide comprises an IgG and IgA Fc region and a region of PD-1 (Programmed cell death protein 1).

Also, the present invention relates to a polynucleotide encoding the Fc-fusion polypeptide of the present invention.

Furthermore, the present invention relates to a vector, such as a viral vector, comprising a polynucleotide encoding the Fc-fusion polypeptide of the present invention.

Still, the present invention relates to a pharmaceutical composition comprising the Fc-fusion polypeptide, polynucleotide or vector of the present invention.

Still, the present invention relates to the Fc-fusion polypeptide, polynucleotide, vector or pharmaceutical composition of the present invention for use in treatment of a cancer.

Still, the present invention relates to a method of treating a cancer in a subject, wherein the method comprises administering the Fc-fusion polypeptide, polynucleotide, vector or pharmaceutical composition of the present invention to a subject in need thereof.

Still furthermore, the present invention relates to a method of preparing the cross*-hybrid Fc-fusion polypeptide targeting or against PD-L1 of the present invention, wherein the method comprises allowing a polynucleotide encoding the cross*-hybrid Fc-fusion polypeptide targeting or against PD-L1 to be expressed to said cross-hybrid Fc-fusion polypeptide in a cell.

Still furthermore, the present invention relates to a method of preparing the vector of the present invention, wherein the method comprises combining a polynucleotide of a vector and the polynucleotide encoding the Fc-fusion polypeptide of the present invention.

Other objects, details and advantages of the present invention will become apparent from the following drawings, detailed description and examples.

The objects of the invention are achieved by polypeptides, polynucleotides, vectors, compositions, uses and methods characterized by what is stated in the independent claims. The preferred embodiments of the invention are disclosed in the dependent claims.

### BRIEF DESCRIPTION OF THE DRAWINGS

FIGS. 1A-E reveal characterization of Adenovirus-ChimericAntibody (Ad-Cab). A) Graphical representation of the IgGA Fc-fusion protein; the cross-isotype Fc is made up of the constant heavy (CH) chains 2 and 3 of an IgA1 (purple) and IgG1 (orange) attached to an PD-1 ectodomain (green) via a glycine linker. The IgGA Fc employs effector mechanism of both an IgG1 and IgA1. B) Schematic representation of oncolytic adenovirus 5/3 delta 24 (Ad5/3 Δ24) constructs with modifications in the E1, E3 and fiber regions. Black inverted triangles represent deletions. Both unarmed Ad5/3 Δ24 (Unarmed) and IgGA PD-L1 Ad-5/3 Δ24 (Ad-Cab) have a 24 base-pair deletion in the E1 region, leading to conditionally replicate in Rb-deficient cells, and a serotype 5 fiber knob with serotype 3 knob and a deletion of the E3B region 14.7k gene. The IgGA PD-L1 fusion protein cassette consisted of cytomegalovirus (CMV) promoter and enhancer and was cloned into the CR1-alpha+gp19k region. C) Quantification of IgGA Fc-fusion proteins over time. A549 20 cells were infected with 100 MOI of Ad-Cab and Unarmed virus and supernatants were collected at different indicated time points. IgGA Fc-fusion proteins were purified, and concentration was assessed by measuring absorbance at 280 nm. D) Competitive assay between Atezolizumab and Ad-Cab. A549 cells were incubated with different concentrations of purified IgGA Fc-fusion proteins from Ad-Cab and followed by addition of 10 μg/ml Atezolizumab. Atezolizumab binding was then analyzed using an PE-labelled anti-human IgG not recognizing IgGA Fc-fusion proteins. E) Representative live cell imaging stills of PBMCs co-incubated with A549 cells at a 10:1, E:T ratio, incubated with or without 10 μg/ml of Atezolizumab or Fc-fusion peptides Ad-Cab. Scale bar 57.88 μm.

FIGS. 2A-E show activation of multiple branches of the immune system. A) The percentage of PD-L1 expression on all cell lines used in the assays. B) FACS-based CDC assay against all five different cell line with Ad-Cab and Unarmed virus. Cells were infected at two indicated MOIs, incubated for 48 hours and pooled serum from healthy volunteers was then added at a final concentration of 15.5%. After 4 hours at 37° C., cell lysis was measured using 7-AAD. ADCC against five different cell lines using either C) PBMCS, D) PMN as effector cells. Indicated viruses were added at 10 and 100 MOI and incubated for 48 hours. Subsequently, PBMCs and PMNs were added at an E:T ratio of 100:1 and 40:1, respectively, and lysis was by quantifying LDH release after 4 hours at 37° C. E) ADCP was measured by incubating target cells with 10 or 100 MOI of Ad-Cab or unarmed virus for 48 hours. After, cells were labeled with CFSE and macrophages were added at a 5:1 (Effector:Ratio) ratio. Phagocytosis was quantified by measuring the uptake of CFSE by macrophages. Levels of significance were set at *p<0.05, ** p<0.01, *** p<0.001, and **** p<0.0001. Error bars represent s.d.

FIGS. 3A-D show PMN's mode of action during ADCC. Gating strategy (left) and histogram (right) of neutrophils incubated alone A), with DiO stained A549 cells B) or DiO stained A549 cells infected with 100 MOI of Ad-Cab C). Trogocytosis of five different cells lines infected at 100 MOI for 48 hours with indicated virus and PMNs added. Neutrophils alone or Neutrophils co-incubated with DiO stained target cells were used as controls (D). PMNs were added at an E:T ratio of 40:1. DiO+PMNs were then calculated using flow cytometry.

FIGS. 4A-C show that activation of multiple branches works in synergy leading to enhanced cytotoxicity. A) Histograms demonstrating the percentage PD-L1 expression on all cell lines used in the assays. Cell lysis of tumor cell lines in the presence B) PMBCs+PMNS and C) PBMCs+PMNs+Serum. PBMCs and PMNs were added at an E:T ratio of 40:1 and 100:1, respectively, while serum was added at 15.5%. Cells were infected with viruses at 100 MOI and incubated for 48 hours or 10 μg/ml of antibody were added 30 minutes prior to adding immune components. Lysis was then detected using an LDH release assay. Levels of significance were set at *p<0.05, ** p<0.01, *** p<0.001, and **** p<0.0001. Error bars represent s.d.

FIGS. 5A-C show results of live-cell killing assays and real time cytotoxicity analysis. A) Representative live-cell killing images of at indicated times. 10⁵ A549 cells were plated and after 15 minutes cells were treated with 3 μM of Incucyte® Caspace3/7 green apoptosis reagent. At one-hour, PBMCs and PMNS were added at 10:1 and 4:1 E:T ratios, respectively, and treated with 10 μg/ml of indicated antibody or Fc-fusion peptides. Scale bars 400 μm. B) Cell death from each indicated treatment. Cell death was measured by counting Caspase3/7+ green spots over the phase confluency normalized at 15 minutes. C) Killing rate (Cell index/min) of Ad-Cab, IgG1-PD-L1 and IgA-PD-L1 with five different cell lines.

FIGS. 6A-E show culturing and characterization of RCC patient derived organoids. A) Representative imaging of renal cancer cell tissue grown in Matrigel as 3D (left) and 2D (right). Immunofluorescence staining of dissociated RCC PDOs using CAIX, Cytokeratin, Vimentin, CD3 and Phalloidin. Scale bar 500 or 200 μm. C) FACS analysis of PD-L1 expression of dissociated RCC PDOs. D) RCC2 PDOs were infected with 5×10⁵ vp of Ad5-RFP Δ24. Cell viability was visualized using Calcein green. Scale bars 200 μm. E) Images of RCC2 PDOs infiltrated by Calcein green stained PBMCs. 10⁵ PBMCs, stained with Calcein green, were added on top of Matrigel and after 4 hours images were taken using an EVOS FL cell imaging system. Scale bars 400 or 200 μm

FIGS. 7A-D reveal efficacy of Ad-Cab with patient derived renal cell cancer organoids. ADCC assays with RCC1 (A), RCC2 (B), RCC3 (C) and RCC4 (D) PDOs. RCC PDOs were infected with viruses at 100 MOI and incubated for 48 hours or 10 μg/ml of antibody were added 30 minutes prior to adding immune components. PBMCs and PMNs were added at an E:T ratio of 40:1 and 100:1, respectively. LDH release assays were performed 4 hours after addition of immune effector cells.

FIG. 8 shows amino acid sequences of the wild type and modified Fc-fusion polypeptides of the present invention. In one embodiment any binding scaffold capable of attaching e.g. the Fc-part of the fusion polypeptide to the PD-L1 can be used in the fusion polypeptide, e.g. between the linker and the IgGA Fc region.

FIGS. 9A-C show Cab vs Cab FT in inducing ADCC with different effector populations. Cells were treated with different concentrations of Fc-fusion peptides and had either PBMCs (A) (100:1, E:T), PMNs (B) (40:1, E:T) or PBMCs+PMNs (C) added for a four incubation. Lysis was then quantified by measuring release of endogenous LDH.

FIGS. 10A-D show whole-blood and mixed leukocyte assay with Cab and Cab-FT. Unmanipulated blood from three donors were treated with 20 μg/ml of Fc-Fusion peptides and incubated for 24 hours. Immune populations were then gated (A) and quantified both percentage (B) and absolute number (C). Dendritic cells and CFSE labeled PBMCs from different donors were incubated with 10 μg/ml of Fc-Fusion peptides or antibody for five days. PBMCs were then collected and CD8+ T cell had their expansion index calculated (D) based on CFSE staining.

FIG. 11A-C show oncolytic fitness and expression of Ad-Cab and Ad-Cab FT. Different types of cells were infected with different indicated MOIs of virus and incubated for three days. Using an MTS assay, cell viability (A) was determined. A549 (B) and B16K1 (C) cells were infected with 100 MOI of virus and amount of Fc-fusion peptides were measured using a HIS-Tag ELISA.

FIG. 12 shows Ad-Cab and Ad-Cab FT ADCC in the presence of PBMCs+PMNs. Cells were infected at various MOIs with virus and left for 48 hours of incubations. After, PBMCs (100:1, E:T) and PMNs (40:1, E:T) were added. After 4 hours, lysis was measured by measuring release of endogenous LDH.

FIGS. 13A-B show the real-time killing of A549 and B16K1 cells with Ad-Cab and Ad-Cab FT. A549 (A) and B16K1 (B) cells were first seeded for 24 hours. After, viruses were added at 100 MOI (B16K1) and 30 MOI (A549) along with both PBMCs (100:1, E:T) and PMNs (40:1, E:T). Cell index was then measured every 30 minutes for the indicated times.

FIGS. 14A-L show the in-vivo efficacy of Ad-Cab and Ad-Cab FT. (A) Schematic diagram of tumor implantation of B16K1 and treatment schedules. Mice were implanted with 500,000 cells in the right flank and then treated either with PBS (Mock), Ad-5/3 Δ 24, Ad-Cab, Ad-Cab FT and mPD-L1. Tumor growth (B) was then recorded. After mice were sacrificed, NK cell activation (C) and T cell activation (D) were measured with flowcytometry. Fc-fusion bio-distribution was then checked in the tumor (E) and liver (F). (G) Schematic diagram of treatment schedule for mice implanted with 300,000 4T1 cells. Same treatment groups were used with B16K1 mice. Tumor growth (H) was recorded for 17 days. After mice were sacrificed, suppressive immune cell populations MDSC-granulocytic (I) and MDSC-monocytic (J) were analyzed via flow-cytometry. The bio-distribution of Fc-fusion peptides were analyzed in the tumor (K) and liver (L).

FIG. 15A-H show xenograft in vivo efficacy of Ad-Cab and Ad-Cab FT. (A) Schematic representation of treatment schedules given to NOD/SCID mice. Mice were first implanted in the right flank 5×10⁶ A549 cells and 5×10⁶ PBMCs intraperitoneally. Treatment groups were divided in mice receiving Ad-5/3 Δ 24, Ad-Cab or Ad-Cab FT. Before treatment, two mice either implanted with PBMCs or not were sacrificed and human CD3+ and CD45+ cells (B) were analyzed in the peripheral blood. Tumor growth was recorded (C). After mice were sacrificed, NK cell activation (D), T cell activation (E) and T cell exhaustion (F) were analyzed in the tumor microenvironment. Biodistribution of the Fc-fusion peptide was then checked in blood (G), tumor (H) and liver (I).

### SEQUENCE LISTING

SEQ ID NO: 1 shows an embodiment of an amino acid sequence of the Fc-fusion polypeptide of the present invention. In one embodiment any binding scaffold capable of attaching the fusion polypeptide (e.g. the Fc-part of the fusion polypeptide) to the PD-L1 can be used as part of the fusion polypeptide (for example between the linker and the IgGA Fc region).

SEQ ID NO: 2 shows an amino acid sequence of the PD-1 ectodomain peptide used in the present invention.

SEQ ID NO: 3 shows an amino acid sequence of the Fc of IgGA used in the present invention.

SEQ ID NO: 4 shows an embodiment of an amino acid sequence of the modified (DF+TE) Fc-fusion polypeptide of the present invention.

SEQ ID NO: 5 shows an amino acid sequence of the modified (DF+TE) Fc of IgGA used in the present invention.

SEQ ID NO: 6 shows a polynucleotide sequence encoding the Fc region of the fusion polypeptide of the present invention.

SEQ ID NO: 7 shows a polynucleotide sequence encoding the region of PD-1 of the fusion polypeptide of the present invention.

SEQ ID NO: 8 shows a polynucleotide sequence encoding the cross-hybrid Fc-fusion polypeptide targeting or against PD-L1 of the present invention.

SEQ ID NO: 9 shows a polynucleotide sequence encoding the modified (DF+TE) Fc-fusion polypeptide of the present invention.

### DETAILED DESCRIPTION OF THE INVENTION

The Fc region endows antibodies the ability to orchestrate immune effector mechanisms by binding to C1q (complement component 1q) and Fc-receptors, eliciting CDC or ADCC/ADCP, respectively. Unlike the Fab fragment, Fc-domains are conserved among the five existing isotype subclasses (IgA, IgD, IgG, IgE and IgM) allowing antibodies to bind to specific Fc-receptors (Fc-α, Fc-δ, Fc-γ, Fc-ε and Fc-μ respectively). This specific Fc-domains/Fc-receptor interaction mediates the regulation of antibody-induced effector mechanisms irrespective of the antigen binding.

In the clinic, all cancer therapeutic antibodies are of the IgG isotype and predominantly of the IgG1 subtype. This is primarily due to the ability of IgG to activate the complement system and natural killer cells (NK), leading to tumor killing. Yet, IgG fails to efficiently activate the most abundant leukocyte population able to infiltrate solid tumors, neutrophils. This is mostly due to the relatively high expression of inhibitory Fc-γIIB (CD32B) and Fc-γIIIB (CD16B), which do not possess any signaling motif yet has been seen to block the activation of ADCC (Derer, S. et al. 2014, MAbs 6, 409-421). In order to capitalize on such a promising population, IgA antibodies have been used since they bind to the Fc-α receptors, CD89, which are highly expressed on neutrophils, monocytes and macrophages consequently eliciting ADCC or ADCP (Brandsma, A. M. et al. 2019, Front. Immunol. 10; Lohse, S. et al. 2012, J. Biol. Chem. 287, 25139-25150). In addition, the Fc-α mediated activation of neutrophils by IgA antibodies has been shown to be more effective in tumor killing than the Fc-γ mediated effector mechanisms by IgG antibodies in multiple types of cancers (Dechant, M. & Valerius, T. 2001, Critical Reviews in Oncology/Hematology vol. 39 69-77).

The therapeutic use of IgA antibodies is, however, restrained due several reasons. Firstly, their relative short half live in serum compared to IgG. Moreover, their inability to bind to C1q and Fc-γ receptors and thus to activate CDC or ADCC elicited by NK, an immune cell population that does not express Fc-α receptors and is required for tumor clearance.

Recently, an Fc portion of therapeutic antibodies has been engineered to simultaneously activate Fc-γ and α receptors, leading to the activation of the effector mechanisms of both an IgG and IgA antibody (Kelton, W. et al. 2014, Chem. Biol. 21, 1603-1609).

The present invention further discloses a surprisingly efficient cross-hybrid Fc-fusion polypeptide against PD-L1 (Programmed death-ligand 1), wherein the Fc-fusion polypeptide comprises an IgG and IgA Fc region and a region of PD-1 (Programmed cell death protein 1).

As used herein “a Fc region” i.e. a fragment crystallizable region refers to the tail part or region of an antibody that is capable of interacting with or binding to C1q and/or a cell surface receptor called a Fc receptor, and optionally some other proteins of the complement system, thereby activating the immune system such as CDC and/or ADCC/ADCP.

IgG immunoglobulin isotype is the most abundant isotype in human serum. There are four subclasses, IgG1, IgG2, IgG3, and IgG4, which are highly conserved but differ in their constant region, particularly in their hinges and upper CH2 domains. Humans possess two IgA subclasses, IgA1 and IgA2, that differ mainly in the structure of their hinge region and in the number of glycosylation sites. “The IgG and IgA Fc region” comprised in the Fc-fusion polypeptide of the present invention can be a combination of a Fc of any IgG subclass (or any fragment thereof) and either a Fc of IgA1 or IgA2 (or any fragment thereof). In one embodiment IgG is IgG1 and/or IgA is IgA1.

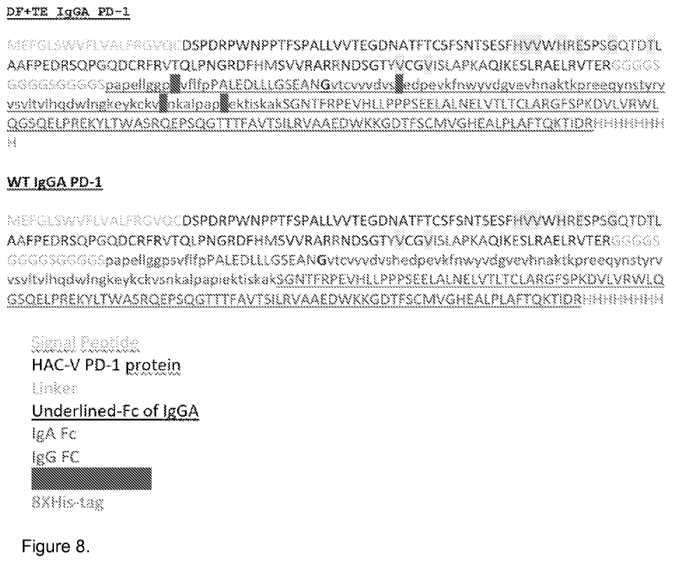

In the Fc-fusion polypeptide of the present invention the Fc region comprises at least parts of both IgG and IgA Fc regions. The Fc region of an IgG as well as IgA comprises two identical peptide fragments i.e. the second (CH2) and third (CH3) constant domains of the antibody's two heavy chains. In one embodiment the IgG and IgA (IgGA) Fc region comprises parts of the constant heavy chain (CH) 2 and/or 3 of an IgG and IgA; parts of the CH2 of IgG1 and the CH3 of IgA1; and/or part of the CH2 of IgG1, part of the CH2 of IgA1, and part of the CH3 of IgA1. In one embodiment the IgG and IgA (IgGA) Fc region comprises amino acids as shown in FIG. 8 or in any of SEQ ID NOs: 1, 3, 4 or 5.

In one embodiment the IgG part (IgG1) of the Fc region comprises CH2 residues PAPELLGGPSVFLFP and/or CH2 (e.g. CHα₂2) residues VTCVVVDVSHEDPEVKFNWYVDGVEVHNAKTKPREEGYNSTYRVVSVLTVLHG DWLNGKEYKCKVSNKALPAPIEKTISKAK. (See e.g. FIG. 8 or any of SEQ ID NOs: 1, 3, 4 or 5.)

In one embodiment the IgA part of the Fc region comprises CH2 (e.g. CHα₁2) residues PALEDLLLGSEAN and/or CH3 (e.g. CHα₁3) residues SGNTFRPEVHLLPPPSEELALNELVTLTCLARGFSPKDVLVRWLQGSQELPREK YLTWASRQEPSQGTTTFAVTSILRVAAEDWKKGDTFSCMVGHEALPLAFTQKTI DR. Optionally, a Gly residue can be inserted e.g. following CH2 (e.g. CHα₁2) to ensure that the length of the grafted loop from CH2 (e.g. CHα₁2) is consistent with that of CHγ₁2. (See e.g. FIG. 8 or any of SEQ ID NOs: 1, 3, 4 or 5.)

In one embodiment the IgG and IgA Fc region of the Fc-fusion polypeptide, such as IgG region, comprises one or more mutations. Mutations can be any mutations known to a person skilled in the art including but not limited to an addition, substitution, or deletion of one or more amino acids. In one embodiment the mutation or mutations of the IgG region is/are selected from the group consisting of H268F, S324T, S239D and 1332E (see e.g. FIG. 8 and/or sequences of SEQ ID NO: 4 or 5). In one embodiment the mutations of the IgG region are H268F, S324T, S239D and 1332E (see e.g. FIG. 8 and/or sequences of SEQ ID NO: 4 or 5). One or more mutations can for example enhance or decrease binding to a receptor and/or the induction of CDC and/or ADCC. In one embodiment, one or more mutations can lead to an increase in IgG effector mechanisms. In one embodiment, one or more mutations can induce higher tumor killing, induce faster tumor cell death, or display better tumor control compared to a corresponding unmutated fusion peptide.

In one embodiment the glycosylation of the Fc region has been modified (e.g. reduced or increased, or the glycosylation type amended). Modification of the glycosylation of the Fc region can have effect e.g. on the binding to the Fc receptor (e.g. Fc-αR) and thus optionally also to the downstream immunological response.

In one embodiment the IgG and IgA Fc region of the Fc-fusion polypeptide is capable of binding one or more Fc-γ receptors (such as Fc-γRIIB and Fc-γRIIIB) and/or a Fc-α receptor. In one embodiment the IgG Fc region binds specifically into Fc-γ receptors (Fc-γR). In humans, three different subgroups of FcγRs have been described: i) FcγRI; ii) FcγRIIA and FcγRIIB; iii) FcγRIIIA and FcγRIIIB. Furthermore, Fc-γRs can be divided into two groups based on whether they activate or inhibit cells from inducing cytotoxic activities. Only one Fc receptor belongs to the FcR-α group, which is called FcαRI.

Binding of the Fc-fusion polypeptide of the present invention to one or more Fc receptors or C1q enables regulation of antibody-induced effector mechanisms. In one embodiment the Fc-fusion polypeptide is capable of eliciting NK-mediated antibody-dependent cell cytotoxicity (ADCC); and/or neutrophil-mediated ADCC, complement-dependent cytotoxicity (CDC) and/or antibody-dependent cell phagocytosis (ADCP). ADCC, CDC and/or ADCP can be determined, predicted or evaluated e.g. with one or more methods or assays as described in the materials and methods sections of the examples of the present disclosure (Complement Dependent Cytotoxicity assay, Antibody Dependent Cell Cytotoxicity Assays, Antibody Dependent Cell Phagocytosis, Trogocytosis, Real-Time quantative analysis, Live-cell killing assay, or live cell imaging, or any combination of said methods or assays, for example).

In a specific embodiment, the inventors of the present disclosure were able to demonstrate that the Fc-fusion peptide of the present invention can induce effector mechanisms of both an IgG, CDC and ADCC with PBMCs, and of an IgA, ADCC with PMNs. These additive effector mechanisms increased tumor killing e.g. when compared to the FDA approved IgG, Atezolizumab, containing an N298A mutation abrogating Fc-γ binding.

In one embodiment the IgGA used in the present invention does not bind to the Fc-neonatal receptor restricting its half-life in the blood and possible toxicity.

In one embodiment the IgG and IgA Fc region of the Fc-fusion polypeptide is connected to the region of PD-1 optionally via a linker such as a glycine linker. A linker can enable a stable and/or bioactive fusion polypeptide. Suitable linkers for the polypeptide of the present invention include but are not limited to one or more of the following: threonine (Thr) based linkers, serine (Ser) based linkers, proline (Pro) based linkers, glycine (Gly) based linkers, aspartic acid (Asp) based linkers, lysine (Lys) based linkers, glutamine (Gln) based linkers, asparagine (Asn) based linkers, alanine (Ala) based linkers, arginine (Arg) based linkers, phenylalanine (Phe) based linkers, glutamic acid (Glu) based linkers, KESGSVSSEQLAQFRSLD and EGKSSGSGSESKST, (Gly)₈, (Gly)₆, GSAGSAAGSGEF, and (GGGGS)₄. In one embodiment the linker comprises at least 50% of naturally encoded amino acids. One suitable glysine linker GGGGSGGGGSGGGGS is shown e.g. in FIG. 8.

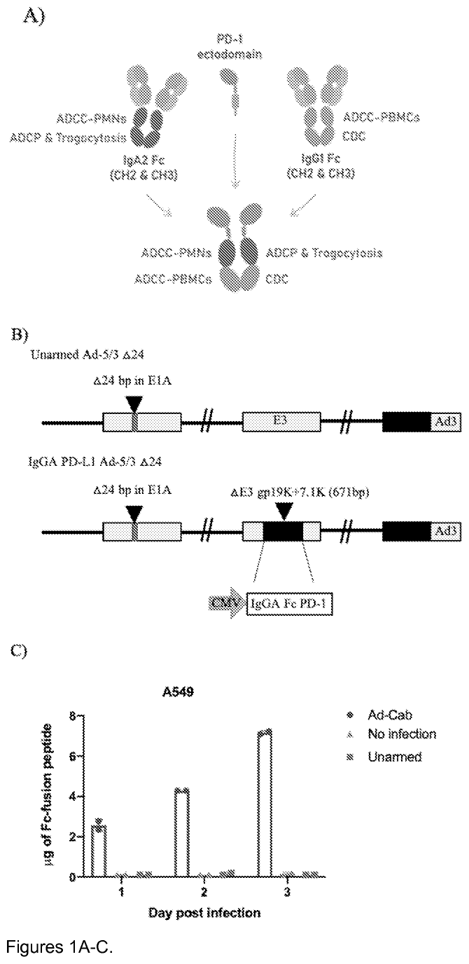

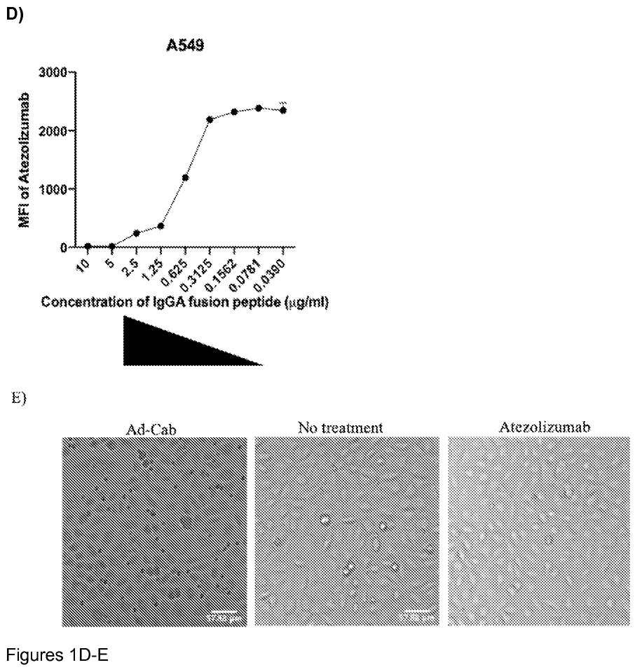

According to the specific design of the Fc-fusion peptide polypeptide of the present invention the IgGA Fc region is connected to a region of PD-1 such as a PD-1 ectodomain or a part thereof (see e.g. FIG. 1B or FIG. 8). In one embodiment said region of PD1 is able to bind to PD-L1.

PD-1, also known as Programmed cell death protein 1 or CD279, is an immune checkpoint cell surface receptor that belongs to the immunoglobulin superfamily. PD-1 has a role in regulating the immune system's response to the cells of the human body by down-regulating the immune system and promoting self-tolerance by suppressing T cell inflammatory activity. PD-1 is known to promote apoptosis (programmed cell death) of antigen-specific T-cells in lymph nodes and reduce apoptosis in regulatory T cells (anti-inflammatory, suppressive T cells). Therefore, PD-1 prevents the immune system from killing cancer cells. An example of a human PD-1 polypeptide sequence is presented with the GenBank accession number AAC51773.1. In humans PD-1 is encoded by the PDCD1 gene. Examples of human PDCD1 gene and mRNA sequences are presented by the sequences with GenBank accession numbers L27440.1 and U64863.1, respectively.

PD-1 binds two ligands, transmembrane proteins PD-L1 and PD-L2. The binding of PD-L1 to the inhibitory checkpoint molecule PD-1 reduces the proliferation of antigen-specific T-cells in lymph nodes, while simultaneously reducing apoptosis in regulatory T cells. An example of a human PD-L1 polypeptide sequence is presented with the GenBank accession number AAF25807.1. In humans PD-L1 is encoded by the CD274 gene. An example of a human CD274 mRNA sequence is presented by the sequence with GenBank accession number AF177937.1.

In one embodiment the region of PD-1 of the Fc-fusion polypeptide comprises or is a PD-1 ectodomain or a part thereof. As used herein “a PD-1 ectodomain” refers to a domain of a membrane protein that comprises extracellular part of the protein (the part outside of a cell). In one embodiment an ectodomain contacts with surfaces enabling signal transduction. PD-1 ectodomain can for example comprise the following amino acids of contact consensus sites: VLNYRMSNQTDKADQGQVHMRYLASLAPKAE. In one embodiment the region of PD-1 ectodomain used in the present invention comprises amino acids shown in FIG. 8 or SEQ ID NO: 2. In a specific embodiment the region of PD-1 or the region of PD-1 ectodomain comprises one or more mutations (e.g. 1-15 or 5-10 mutations, such as 9 mutations) that optionally increase its affinity towards PD-L1 compared to a region of PD-1 or an ectodomain without said one or more mutations. (See e.g. FIG. 8 or any of SEQ ID NOs: 1, 2, or 4.)

In one embodiment of the invention any binding scaffold capable of attaching the fusion polypeptide (e.g. the Fc-part of the fusion polypeptide) to the PD-L1 can be used as part of the fusion polypeptide (for example between the linker and the IgGA Fc region).

In one embodiment the Fc region of the Fc-fusion polypeptide comprises an amino acid sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 3 or 5; the region of PD-1 comprises an amino acid sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 2; and/or
the cross-hybrid Fc-fusion polypeptide targeting or against PD-L1 comprises an amino acid sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 1 or 4.

In the present disclosure, the term “polypeptide” refers to polymers of amino acids of any length. As used in the present disclosure, the terms “polypeptide”, “peptide” and “protein” are used interchangeably. Furthermore, as used herein “a polynucleotide” refers to any polynucleotide, such as single or double-stranded DNA (genomic DNA or cDNA) or RNA (e.g. mRNA or rRNA), including but not limited to a nucleic acid sequence encoding a polypeptide in question or a conservative sequence variant thereof. Conservative nucleotide sequence variants (i.e. nucleotide sequence modifications, which do not significantly alter biological properties of the encoded polypeptide) include variants arising from the degeneration of the genetic code and from silent mutations. “A fragment of a polypeptide or polynucleotide” refers to a fragment of any length, e.g. any part of a polypeptide or polynucleotide.

The present invention also refers to a polynucleotide encoding the Fc-fusion polypeptide of the present invention. In one embodiment the polynucleotide encoding the Fc region comprises a polynucleotide sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 6;
the polynucleotide encoding the region of PD-1 comprises a polynucleotide sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 7; and/or the polynucleotide encoding the cross-hybrid Fc-fusion polypeptide targeting or against PD-L1 comprises a polynucleotide sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 8 or 9.

Identity of any sequence or fragments thereof compared to the sequence of this disclosure refers to the identity of any sequence compared to the entire sequence of the present invention. As used herein, the % identity between the two sequences is a function of the number of identical positions shared by the sequences (i.e. % identity=# of identical positions/total # of positions×100), taking into account the number of gaps, and the length of each gap, which need to be introduced for optimal alignment of the two sequences. The comparison of sequences and determination of identity percentage between two sequences can be accomplished using mathematical algorithms available in the art. This applies to both amino acid and nucleic acid sequences. As an example, sequence identity may be determined by using BLAST (Basic Local Alignment Search Tools) or FASTA (FAST-AII). In the searches, setting parameters “gap penalties” and “matrix” are typically selected as default. In one embodiment the sequence identity is determined against the full length sequence of the present disclosure.

It is well known that deletion, addition or substitution of one or a few amino acids or polynucleotides does not necessarily change the properties of a polypeptide. Therefore, the invention also encompasses variants and fragments of the polypeptides or polynucleotides of the present invention. The term “variant” as used herein refers to a sequence having minor changes in the amino acid or polynucleotide sequence as compared to a given sequence. Such a variant may occur naturally e.g. as an allelic variant or it may be generated by modification. It may comprise amino acid substitutions, deletions or insertions, but it still functions in substantially the same manner as the given polypeptides.

A “region” or “fragment” of a given polypeptide means part of that polypeptide, e.g. a sequence that has been truncated at the N- and/or C-terminal end, e.g. lacking a signal sequence or a linker sequence.

Transfer of the polynucleotide of the present invention to a cell in vitro, ex vivo and in vivo can be performed by any of the methods that physically or chemically permeabilize the cell membrane. In some embodiments, non-viral delivery is contemplated. Non-viral delivery or polynucleotides includes but is not limited to calcium phosphate precipitation, DEAE-dextran, electroporation, direct microinjection, DNA-loaded liposomes, cell sonication, gene bombardment using high velocity microprojectiles, and receptor-mediated transfection.

In the present invention a fusion of a PD-1 region and a cross-hybrid IgGA Fc region enables unexpected and/or synergistic anti-tumor properties. Furthermore, the fusion polypeptide can be directly expressed into the tumor micro-environment by using vectors such as viral or oncolytic adenoviral vectors. In a specific embodiment vectors can further enhance the cross-hybrid Fc-fusion polypeptide therapy and/or enable circumventing immune-related adverse events. Indeed, the present invention also relates to a vector, such as a viral vector, comprising a polynucleotide encoding the Fc-fusion polypeptide of the present invention.

The term “vector” refers to a nucleic acid compound and/or composition that transduces, transforms, or infects a cell, thereby causing the cell to express nucleic acids and/or polypeptides other than those native to the cell, or in a manner not native to the cell. “A vector” or “an expression vector” contains a sequence of nucleic acids to be expressed by the infected or modified cell. Optionally, the expression vector also comprises materials to aid in achieving entry of the nucleic acids into the cell, such as a viral sequence, liposome, protein coating, or the like. Expression vectors suitable for the present invention include those into which a nucleic acid sequence (i.e. a polynucleotide) can be inserted, optionally along with any preferred or required operational elements. Optionally, expression vectors can be transferred into a cell and replicated therein. Vectors can be linear or circularized and optionally can contain restriction sites of various types e.g. for fragmentation or linearization. In a specific embodiment the vector is a viral vector or a plasmid; and/or the vector is a viral vector, wherein a virus of the viral vector is a member of a family selected from the group comprising Herpesviruses, Poxviruses, Hepadnaviruses, Flavivirus, Togavirus, Coronavirus, Hepatitis D, Orthomyxovirus, Paramyxovirus, Rhabdovirus, Bunyavirus, Filovirus and Retroviruses, and Adenovirus.

In one embodiment the vector is an oncolytic viral vector or an oncolytic adenoviral vector. In one embodiment oncolytic adenoviral vectors minimalize unwanted cytotoxicity of the therapeutic agents of the present invention. The oncolytic adenoviruses of the present invention were able to secrete the cross-hybrid IgGA Fc-fusion peptides able to bind to PD-L1 and activate multiple immune pathways, not activated when IgG or IgA antibody is added alone, resulting in enhanced tumor killing. Adenoviral vectors of the present invention are able to both i) avoid the limiting factor of immune exhaustion by activating all possible immune mechanisms and ii) to avoid the severe grade 3 and 4 adverse events by expressing the IgGA-Fc fusion peptide only in the tumour microenvironment.

As used herein “an oncolytic viral vector” refers to a viral vector that infects and/or kills tumor or cancer cells selectively. As used herein “an oncolytic adenoviral vector” thus refers to an adenoviral vector capable of infecting and killing cancer cells by selective replication in tumor versus normal cells. In one embodiment of the invention, the adenoviruses are capable of replicating and killing cancer cells while diverting the anti-viral immune response against the tumor. In one embodiment adenoviruses used in the present invention may be of any type and species of adenoviridae (e.g. not limited to human adenovirus) such as those suitable for treating a human or an animal. Alternatively, various types of adenoviral vectors can be used according to the present invention. The backbone of the adenoviral vector may be of any serotype or a combination thereof. In one embodiment the oncolytic adenoviral vector is selected from an Ad26, Chimp Ad, Gorilla Ad, Ad5, Ad3 or Ad5/3 vector selected from an Ad26, Chimp Ad, Gorilla Ad, Ad5, Ad3 or Ad5/3 vector. As an example, “Ad5/3 vector” refers to a chimeric vector having parts of both Ad5 and Ad3 vectors. For example, in one embodiment the vector is an adenoviral Ad5/3 vector comprising an adenovirus serotype 5 (Ad5) nucleic acid backbone and an adenovirus serotype 3 (Ad3) fiber knob.

The vectors of the present invention can be modified in any way known in the art, e.g. by deleting, inserting, mutating or modifying any amino acids or amino acid regions. The vectors can be made tumor specific with regard to replication. For example, the adenoviral vector may comprise modifications in E1, E3 and/or E4 such as insertion of tumor specific promoters, deletions of areas and insertion of one or more transgenes.

In one embodiment of the invention the vectors are replication competent only in cells, which have defects in the Rb-pathway, specifically Rb-p16 pathway. These defective cells include all tumor cells in animals and humans. As used herein “defects in the Rb-pathway” refers to mutations and/or epigenetic changes in any genes or proteins of the pathway. A tumor specific oncolytic adenovirus may be engineered for example by deleting 24 base pairs (D24) of the constant region 2 (CR2) of E1. As used herein “D24” or “24 bp deletion” refers to a deletion of nucleotides corresponding to amino acids 122-129 of the vector according to Heise C. et al. (2000, Nature Med 6, 1134-1139). In one embodiment of the invention the adenoviral vector comprises a E1 gene deletion e.g. the 24 bp deletion (oncolytic virus) of the E1 gene. E1 gene deletion may be partial or total deletion of the E1 region.

In one embodiment the vector is an oncolytic adenoviral vector comprising one or more of the following: capability to conditionally replicate only in tumour cells with a deficient Rb-pathway; a 24 bp deletion (D24) in the Rb binding constant region 2 of adenoviral E1A; a deletion in the E3 area, optionally a deletion of viral CR1-alpha+gp19K region in the E3A area; a deletion in the E3B area, optionally a deletion of viral 14.7K region in the E3B area.

In one embodiment the polynucleotide encoding the Fc-fusion polypeptide is in the E3A gene in the place of the deleted area of E3, optionally under a tumor specific promoter. In one embodiment the polynucleotide encoding the Fc-fusion polypeptide, optionally under a tumor specific promoter, is in the place of a deleted CR1-alpha+gp19K region in the E3A area. FIG. 1B shows one adenoviral vector, wherein the Fc-fusion peptide has been cloned in the CR1-alpha+gp19K region of the E3A gene region.

Insertion of exogenous elements may enhance effects of vectors in target cells. The use of exogenous tissue or tumor-specific promoters is common in recombinant vectors and they can also be utilized in the present invention. Suitable promoters are well known to a person skilled in the art. The viral vectors utilized in the present inventions may also comprise other modifications than described above.

Any additional components or modifications may optionally be used but are not obligatory for the present invention.

The present invention further concerns a method of preparing the vector of the present invention, wherein the method comprises combining a polynucleotide of a vector and the polynucleotide encoding the Fc-fusion polypeptide of the present invention. Conventional methods of preparing recombinant polynucleotides, or vectors or plasmids comprising said polynucleotides to be expressed by the infected or modified cell are known to a person skilled in the art.

The present invention further concerns a method of preparing the cross-hybrid Fc-fusion polypeptide targeting or against PD-L1, wherein the method comprises allowing a polynucleotide encoding the cross-hybrid Fc-fusion polypeptide targeting or against PD-L1 to be expressed to said cross-hybrid Fc-fusion polypeptide in a cell. In one embodiment the method comprises introducing the nucleic acid molecule, polynucleotide or vector of the present invention into a cell and thereafter allowing production of the fusion polypeptide by said cell, and optionally further determining the produced fusion polypeptide. In another embodiment the method comprises transfecting or transducing a plasmid comprising the polynucleotide of the present invention into a cell and thereafter allowing production of the fusion polypeptide by said cell, and optionally further determining the produced fusion polypeptide. Conditions permitting expression or production of the fusion polypeptide include but are not limited to conditions allowing survival and/or division of cells suitable for production of the fusion polypeptide. As used herein “a cell” refers to any cell capable of allowing expression or production of the polypeptide of the present invention from a polynucleotide encoding said polypeptide. In one embodiment the cell is an animal cell, mammalian cell, human cell, bacterial cell, fungal cell, or plant cell.

A pharmaceutical composition is also comprised within the scope of the present invention. Such pharmaceutical compositions comprising the Fc-fusion polypeptide, polynucleotide and/or vector of the present invention may also comprise any other therapeutically effective agents, any other agents, such as a pharmaceutically acceptable solvent, diluent, carrier, buffer, excipient, adjuvant, carrier medium, antiseptic, filling, stabilizing or thickening agent, and/or any components normally found in corresponding products. In one embodiment the polypeptide, polynucleotide and/or vector are in one or more compositions comprising a pharmaceutically acceptable carrier.

The pharmaceutical composition may be in any form, such as in a solid, semisolid or liquid form, suitable for administration. A formulation can be selected from the group consisting of, but not limited to, for example powder, solutions, emulsions, suspensions, spray, tablets, pellets and capsules. Means and methods for manufacturing or formulating the present pharmaceutical compositions or preparations are known to persons skilled in the art. The pharmaceutical compositions may be produced by any conventional processes known in the art.

The Fc-fusion polypeptide, polynucleotide, vector or pharmaceutical composition of the present invention can be used for treatment of a cancer. Also, the present invention relates to a method of treating a cancer in a subject, wherein the method comprises administering the Fc-fusion polypeptide, polynucleotide, vector or pharmaceutical composition to a subject in need thereof.

In a specific embodiment of the invention the polypeptide, polynucleotide, vector or pharmaceutical composition is (to be) administered to a patient, or a cell is transformed or transfected with a polynucleotide or vector of the invention. Amounts and regimens for therapeutic administration of the polypeptide, polynucleotide, vector or pharmaceutical composition of the present invention can be determined readily by those skilled in the clinical art of treating cancers. Generally, the dosage of the polypeptide or polynucleotide varies depending on multiple factors such as age, gender, other possible treatments, a cancer in question and severity of the symptoms. Therapeutically effective amounts of compounds can be empirically determined using art-recognized dose-escalation and dose-response assays. For instance, when viral vectors are (to be) used for polynucleotide delivery those skilled in the art are well aware of how to apply gene delivery to in vivo and ex vivo situations. For viral vectors, one generally will prepare a viral vector stock. Depending on the kind of virus and the titer attainable, the vector is typically administered, optionally in a pharmaceutically acceptable carrier, in an amount of 10⁴ to 10¹³ viral particles, e.g. in an amount of at least 10⁷, 10⁸, 10⁹ or at least 10¹⁰ viral particles. Similar figures may be extrapolated for liposomal or other non-viral formulations by comparing relative uptake efficiencies. Monitoring the progression of the therapy or patient side effects can provide additional guidance for an optimal dosing regimen.

In one embodiment of the invention a subject is a human, a child, an adolescent or an adult. Also any animal or mammal, such as a pet, domestic animal or production animal, suffering from a cancer or tumor may be a subject of the present invention. The mammal can be selected e.g. from the group comprising a human, common chimpanzee, monkey, mouse, rat, hamster, rabbit, dog, and cat. A subject is in need of a treatment or prevention of a cancer or tumor, or a subgroup thereof, with the polypeptide, polynucleotide, vector or pharmaceutical composition of the present invention.

Before classifying a subject or patient as suitable for the therapy of the present invention, the clinician may for example study the subject or patient or study any symptoms or assay any disease markers of the subject. Based on the results deviating from the normal or revealing a disease such as a cancer or tumor, the clinician may suggest methods of treatment of the present invention for the subject. In one embodiment the subject to be administered with the therapeutic(s) of the present invention has been diagnosed with a cancer or tumor.

Administration of the polypeptides, polynucleotides, vectors and/or pharmaceutical compositions can be conducted through any suitable method known to a person skilled in the art. Indeed, any conventional method may be used for administration of the polypeptide, polynucleotide, vector or pharmaceutical composition of the present invention to a subject. The route of administration depends on the formulation or form of the composition, the disease, the patient, and other factors. In one embodiment of the invention, the administration is conducted through an intratumoral, intramuscular, intra-arterial, intravenous, intrapleural, intravesicular, intracavitary, or intraperitoneal injection, or an oral administration. In one embodiment of the invention the polypeptide, polynucleotide, vector or pharmaceutical composition is administered systemically. It is also possible to combine different routes of administration. A variety of direct, local and regional administration approaches may be taken. For example, a tumor or cancer can be directly injected with the polypeptide, polynucleotide, vector or pharmaceutical composition of the present invention. The polypeptides, polynucleotides, vectors and/or pharmaceutical compositions may also be used together (simultaneously or sequentially) with other therapeutic agents or therapeutic methods or a combination of treatments. For example, the method or use of the invention may further comprise radiotherapy, chemotherapy, administration of other drugs and/or any clinical operations. In one embodiment a tumor or cancer can be treated only with a therapeutic agent or agents, optionally in combination with a surgery, chemotherapy and/or radiotherapy, e.g. prior to, during and/or after a surgery, chemotherapy and/or radiotherapy.

A desired dosage can be administered in one or more doses at suitable intervals to obtain the desired results. Only one administration of the polypeptide, polynucleotide, vector or pharmaceutical composition may have therapeutic effects, but specific embodiments of the invention require several administrations during the treatment period. In one embodiment the polypeptide, polynucleotide, vector or pharmaceutical composition are (to be) administered one or several times during the treatment period. For example, administration may take place from 1 to 30 times, 1 to 20 times, 1 to 10 times, two to eight times or two to five times in the first 2 weeks, 4 weeks, monthly or during the treatment period. The length of the treatment period may vary, and may, for example, last from a single administration to 1-12 months, two to five years or even more.

Administration of the peptides, polynucleotides, vectors or pharmaceutical compositions of the present invention may precede or follow the other agent treatment, by intervals ranging from minutes to weeks. In embodiments where the other agent and the peptides, polynucleotides, vectors or pharmaceutical compositions of the present invention are administered separately, one would generally ensure that a significant period of time did not expire between the time of each delivery, such that the other agent and the therapeutic of the present invention would still be able to exert an advantageously combined effect. In such instances, one could administer both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Treatments with one or more agents can be repeated.

Any method or use of the present invention may be carried out either in vivo, ex vivo or in vitro.

Any tumor or cancer, which can be treated, which progress can be slowed down or wherein the symptoms can be ameliorated, is included within the scope of the present invention. In one embodiment the immune response is directed against a tumor (including malignant and/or benign tumors as well as primary and/or secondary tumors) and/or cancer (i.e. primary and/or secondary malignant neoplasia).

Any tumor or cancer can be a target of the polypeptide, polynucleotide, vector or pharmaceutical composition of the present invention. In one embodiment of the invention, the cancer is selected from the group comprising or consisting of nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer, and any combination thereof.

As used herein, the term “treatment” or “treating” refers to administration of at least the polypeptide, polynucleotide, vector and/or pharmaceutical composition of the present invention to a subject. The term “treating”, as well as words stemming therefrom, as used herein, do not necessarily imply 100% or complete response. Rather, there are varying degrees of which one of ordinary skill in the art recognizes as having a potential benefit or therapeutic effect. In this respect, the methods and uses of the present invention can provide any degree of treatment or prevention of a disease such as a cancer or tumor. Therefore, “treating” includes not only complete cure but also for example prophylaxis, amelioration, or alleviation of disorders or symptoms related to a disease in question, such as a cancer or tumor. Therapeutic effect may be assessed by any method known to a person skilled in the art, for example by monitoring the symptoms of a patient, the size or shape of the tumor or cancer, or markers e.g. in a tumor or cancer or in blood.

The polypeptides, polynucleotides, vectors and/or pharmaceutical compositions can be administered to a subject in a therapeutically effective amount. As used herein, the term “therapeutically effective amount” refers to an amount of polypeptides, polynucleotides, vectors and/or pharmaceutical compositions with which the harmful effects of a disease or disorder (e.g. cancer or tumor) are, at a minimum, ameliorated. The harmful effects include any detectable or noticeable effects of a subject such as pain, headaches, dizziness, fever, persistent cough, a change in bowel habits, a change in urination, indigestion or difficulty in swallowing, bloating, blood in the stool, unexplained anemia, unexplained persistent lumps of tissues, swollen glands, hoarseness, change in a mole, unexpected weight loss, and/or nonhealing sores.

The present invention further concerns a method of increasing CDC and/or ADCC/ADCP in a subject, wherein the method comprises carrying the polynucleotide of the present invention to a target cell or tissue, expressing the fusion polypeptide encoded by the polynucleotide in the cell, and increasing CDC and/or ADCC/ADCP in said target cell or tissue.

It will be obvious to a person skilled in the art that, as the technology advances, the inventive concept can be implemented in various ways. The invention and its embodiments are not limited to the examples described below but may vary within the scope of the claims.

### EXAMPLES

### Example 1

### Materials and Methods

### Cell Lines and Antibodies

Human lung cancer cell line A549, human breast cancer cell line MDA-MB-436, murine breast cancer cell line 4T1 and murine skin cancer cell lines B16F1 and B1610 were purchased from the American Type Culture Collection (ATCC). All cell lines were cultured under appropriate conditions and regularly checked for mycoplasma contaminations. Atezolizumab and IgG1-PD-L1 were purchased from Invivogen. IgA-PD-L1 was kindly provided by Dr. Jeanette Leusen of Utrecht University Medical University.

### Preparation of Conditionally Replicating Adenovirus and Transgene Modifications

All adenoviruses were generated as conditionally replicating adenoviruses using standard protocols previously described (Kanerva, A. et al. 2003, Mol. Ther. 8, 449-458). Ad-Cab, Ad-Cab FT and unarmed viruses are of the chimeric 5/3 serotype with a 21-nucleotide deletion in the E1A region resulting in selective replication in Rb-deficient pathway cells. Ad-RFP has the same genetic modification in E1A but originates from the serotype 5. All transgenes were cloned by replacing the gp19K+7.1K region in the E3 gene. Transgenes were inserted using an inhouse method not yet published.

### Generation of Fc-Fusion Peptide

The Fc-Fusion peptide consists of a chimeric Fc containing constant domains of an IgG1 and IgA connected to an enhanced PD-1 ectodomain via five GGGS linkers. The cross-hybrid Fc has been described (Kelton, W. et al. 2014, Chem. Biol. 21, 1603-1609) as well as the PD-1 ectodomain (Maute, R. L. et al. 2015, Proc. Natl. Acad. Sci. U.S.A 112, E6506-E6514).

Amino acid sequences of the wild type and modified Fc-fusion polypeptides are shown in FIG. 8 and SEQ ID NOs: 1-5.

### Cell Viability Assays

10,000 cells were plated in a 96-well plate overnight and subsequently infected at different MOIs. Three-days post infections cell viability was determined by MTS according to the manufacturer's protocol (Cell Titer 96 AQueous One Solution Cell Proliferation Assay; Promega, Nacka, Sweden). Spectrophotometric data were acquired with Varioskan LUXMultimode Reader (Thermo Scientific, Carlsbad, CA, USA) operated by Skanltsoftware.

### Competition Assay

100,000 A549 cells were plated in a 96-well, washed with PBS and incubated with various concentrations of purified Fc-fusion peptide for 45 minutes on ice. Next, 10 ug/ml of Atezolizumab (Invivogen) was added and incubated for 30 minutes on ice. Atezolizumab was then detected by staining with a PE labelled anti-human IgG (Biolegend). Cells where then washed and resuspended in PBS. Competition was then quantified by flow cytometry using the BD Accuri 6 plus (BD Biosciences) and analyzed using the FlowJo software (Tree Star, Ashland, OR, USA).

### Serum Collection

40 mls of blood was collected from healthy volunteers in BD Vacutainer collection tubes (BD Bioscience) and allowed to clot for 30 minutes at room temperature. After clotting, clots were removed and samples centrifuged for 5 minutes at 2500 rpm. Separated Serum was collected and samples from 15 volunteers were pooled together. Pooled samples were then aliquoted, stored at −80° C. and thawed when required.

### PBMC and PMN Isolation

PMNs and PBMCs were isolated from buffy coats as previously described (Kelton, W. et al. 2014, Chem. Biol. 21, 1603-1609). In short, buffy coats were diluted in PBS (1:1) and then layered on top of a double density layer consisting of Histopaque 1199 (Sigma Aldrich) and Ficoll-plaque PLUS 1.077 g/mL (GE Healthcare). Samples were centrifuged at 400 g for 30 minutes with minimum acceleration and no breaks. The PBMC and PMN layers were subsequently removed between serum and Ficoll or in the Histopaque layer, respectively. Cells were cultured in 1×RPMI (Roswell Park Memorial Institute) supplemented with 10% Fetal bovine serum (FBS, Gibco), 2 mM Glutamax (Gibco) and 1% Penicillin-Streptomycin (Gibco).

### Complement Dependent Cytotoxicity Assay

100,000 cancer cells were plated per well to a 96-well plate and infected with 10 or 100 MOI of virus for 48 hours at 37° C. After, complement active pooled human serum or heat inactivated serum (by incubating serum at 56° C. for 30 minutes) was added to a final concentration of 15.5% and incubated for 4 hours. Subsequently, lysis was quantified by washing cells and stained with 7-amino-actinomycin D (7-AAD) (eBioscience) and measured by flow cytometry.

### Antibody Dependent Cell Cytotoxicity Assays

ADCC assays were performed through measuring cell killing by determining the amount of LDH released using a colometric assay (CyQUANT™ LDH Cytotoxicity Assay). Prior the assays, the optimal cells seeded was determined by measuring the spontaneous release of LDH of untreated cells or cells treated with 1% Triton. Cells were then seeded at their optimal number and infected with 10 or 100 MOI of virus for 48 hours at 37° C. Afterwards, PBMCs or PMNs were added in a 100:1 or 40:1 ratio (E:T), respectively, and incubated for 4 hours at 37° C. LDH was measured using the mentioned kit and percent cytotoxicity was calculated as follows: Percent cytotoxicity=(“experimental”−“effector plus target spontaneous”)/(“target maximum”−“target spontaneous”)×100%, where “experimental” corresponds to the signal measured in a treated sample, “effector plus target spontaneous” corresponds to the signal measured in the presence of PMN or PBMC and tumor cells alone, “target maximum” corresponds to the signal measured in the presence of detergent lysed tumor cells.

### Antibody Dependent Cell Phagocytosis

Around 2×10⁶ freshly isolated PBMCs were cultured in a T25 culture flask for two hours at 37° C. Floating cells were removed, and adherent monocytes were differentiated into macrophages by culturing in RPMI supplemented with 50 μg/ml of MCSF (Sigma Aldrich) for 7 days at 37° C.

1×10⁴ cells were incubated and infected with indicated viruses at 10 and 100 MOI for 48 hours. Cells were then labelled with CFSE (ThermoFisher), according to the manufactures instructions and monocyte-differentiated macrophages were added at a 5:1 Effector: Target ratio. After four hours, supernatant containing macrophages were removed and CFSE was measured using flow cytometry.

### Trogocytosis

Trogocytosis was performed as previously described (Treffers, L. W. et al. 2020, Cancer Immunol. Res. 8, 120-130). In brief, 5,000 cells were infected with 100 MOI of virus and incubated for 48 hours at 37° C. Cell's lipid membrane were labeled with 5 μm of DiO (SantaCruz), a lipophilic membrane dye, for 30 minutes at 37° C. Cells were washed and incubated with PMNs at a 40:1 (E:T) ratio. Samples were fixed using Paraformaldehyde (Sigma Aldrich) and measured using flow cytometry. Trogocytosis was measured by firstly gating on the neutrophil population and measuring the mean fluorescent intensity of cells positive for DiO.

Real-Time Quantative Analysis (xCelligence Assay)

The ability to induce ADCC was analysed using the impedance-based real-time cytotoxicity assay with the XCELLigenc system (ACEA Biosceinces, San Diego, CA, USA). In each well 25,000-100,000 cells were plated for 24 hours. 5 μg/ml of designated antibody or purified Fc-fusion peptide was added along with PBMCs and PMNs at a 10:1 and 4:1 Effector: Target ratio. Cell index was measured every minutes for a period of six hours. Killing rate was obtained by constructing a linear trendline and calculating the slope.

### Live Cell Imaging

Imaging target to effector cell contacts, 15,000 A549 cells were plated per well of a 24 well plate (Corning) overnight. Cells were imaged for 30 minutes and subsequently treated with 10 μg/ml of indicated Fc-fusion peptides and PBMCs were added at 10:1 E:T ratio. The videos were acquired using an ANDOR Spinning Disc Microscope equipped with a Zyla camera (SR Apochromat×100 objective, NA 1.49). Images were acquired every 5 min over the course of 2 h20 min.

Live-cell killing assay was performed by plating 100,000 A549 cells per well of a 24 well plate (Corning) overnight. Fifteen minutes prior imaging, cells were incubated with 3 μM of Incucyte Caspace3/7 green apoptosis assay reagent (Sartorius). Cells were imaged using the IncuCyte S3 live cell analysis system equipped with a 10× air objective for a total of 24 hours. Images were acquired every 15 minutes and 4 fields of view were imaged per well. After one-hour of imaging, cells were treated with indicated antibodies at 5 μg/ml and PMNs and PBMCs were added at 100:1 and 40:1 E:T ratios, respectively. Treated cells were returned to the IncuCyte S3 and imaged for the remainder 23h. Videos were processed with the IncuCyte analysis software and are displayed as 4 fields of view per second.

### Renal Cell Carcinoma Patient Derived Samples and Ethical Considerations

Renal Cell Carcinoma samples were obtained from four patients that underwent surgical removal of the tumors. Tumor samples were collected and delivered directly from the Peijas Hospital. This study was approved by the Helsinki University Hospital Ethical committee (Renal Cell Carcinoma patients DNRO 115/13/03/02/15). The study was conducted in accordance with the declaration of Helsinki and patients gave their written consent.

### Renal Cell Carcinoma Patient Derived Organoid Culturing

Frozen disassociated cells were grown in DMEM/F12 media in 30% Matrigel (Corning) on ultralow attachment plates (ULA Corning). Cells were split and washed with Gentle cell disassociation media (Stemcell) and 10000 cells mixed with 30% Matrigel and grown for 1 week before the experiment. DMEM/F12 media was supplemented with 5% FBS, 8.4 ng/ml of Cholera toxin (Sigma), 0.4 μg/ml Hydrocortisone (Sigma), 10 ng/ml Epidermal Growth Factor (Corning®), 24 μg/ml Adenine (Sigma), 5 μg/ml Insulin (Sigma) and 10 μM of Y-27632 RHO inhibitor (Sigma).

### Immunofluorescence and Flow-Cytometry on Renal Cell Carcinoma Patient Derived Organoids

Gentle Cell Disassociation media used on organoid cultures, cells washed and carefully pipetted to disassociate cells. Cells were plated on 8 well Nunc; Lab-Tek; II Chamber Slides, and cultured for 4 days. Cells were fixed in 4% cold paraformaldehyde and stained with CAIX (2D3), Vimentin (2D1) or Cytokeratin pan (AE-1/AE-3) antibodies (Novus Biologicals) or Alexa Fluor 633 Phalloidin (Thermo Fisher Scientific) Microscopy pictures were taking using an EVOS FL cell imaging system (Thermo Fisher Scientific).

Ad-RFP Infection and PBMC Co-Culture with Renal Cell Carcinoma Patient Derived Organoids

100,000 VPs of Ad5-RFP were added on top of the media of already cultured organoids and RFP expression was then monitored. Cell viability of PDOs was monitored by adding 1 μM of Calcein AM (Thermo Fisher Scientific, C1430).

As for co-culturing, 15,000 isolated PBMCs were first stained with 1 μM Calcein AM and added on top of the media of RCC PDOs and cultured for 4 hours at 37° C. PBMC invasion was then visualized using the EVOS FL cell imaging system.

Antibody Dependent Cell Cytotoxicity Assays with Renal Cell Carcinoma Patient Derived Organoid

RCC PDOs were infected with viruses at 10 or 100 MOI by adding it on top of the supernatant media and incubated for 72 hours at 37° C. PBMCs and PMNs were then added individually or combined at 100:1 and 40:1 (E:T), respectively. The number of cells in the organoids were assumed to be 10,000. After 4 hours of incubation at 37° C., cell killing was measured by determining the amount of LDH released using a colometric assay (CyQUANT™ LDH Cytotoxicity Assay). Percent cytotoxicity was then calculated as stated before.

### Statistical Analysis

Statistical analysis was performed using GraphPad Prism 7 (GraphPad Software, Inc., La Jolla, CA, USA). Data was analyzed using an unpaired t-test where n≥3. Levels of significance were set at *p<0.05, ** p<0.01, *** p<0.001, and **** p<0.0001. Error bars represent s.d.

### Results

### Characterization of Oncolytic Viruses Expressing IgGA-Chimeric Anti-PD1 Fc-Fusion Peptides

In this study, we generated an oncolytic adenovirus, Ad-Cab (AdenovirusChimericAntibody, Ad-Cab) expressing a chimeric IgG-IgA (IgGA) Fc linked to an enhanced PD-1 ectodomain via a glycine linker, able to bind to PD-L1 (FIG. 1a). The Fc-fusion peptide was cloned in the gp19K+7.1K region of the E3A gene (FIG. 1b).

Firstly, we assessed the amount of Fc-fusion peptide secreted at different time points of infection. After 1 day of infection in A549 cells, Ad-Cab secreted approximately 2 μg of the Fc-fusion peptide and production kept increasing till day 3 reaching 7 μg (FIG. 1c). To assess whether the produced Fc-fusion peptide could bind to PD-L1 we performed a competition assay with a commercially available anti-PD-L1 (Atezolizumab), a well-established binder of PD-L1 and disruptor of the PD-1/L1 axis. To this end we co-incubated A549 cells with increasing concentrations of Fc-fusion peptides, purified from the supernatant of infected cells, followed by the addition of 10 μg/ml of Atezolizumab. Detection of bound Atezolizumab to PD-L1 was then analyzed by adding a secondary PE labelled anti-human IgG not able to recognize the Fc-fusion peptide. When no Fc-fusion peptide was added Atezolizumab was able to bind to PD-L1 (FIG. 1d). Yet, as the concentration of both Fc-fusion peptides increased, Atezolizumab binding decreased substantially (FIG. 1d). Moreover, to further demonstrate PD-L1 binding we conducted live cell imaging to observe whether the Fc-fusion peptide could mediate close cell-contacts when PBMC were co-incubated with lung carcinoma A549 cells. When Ad-Cab was added, peripheral blood mononuclear cells (PBMCs) were shown to be in clear proximity to A549 compared to when we added Atezolizumab, a clinical PD-L1 antibody holding a N298A mutation abrogating Fc-binding (FIG. 1f). Taken together, we demonstrated that Ad-Cab is able to secrete high levels of Fc-fusion peptides able to bind to PD-L1 and outcompete Atezolizumab.

### The Secreted Fc-Fusion Peptides Activate Effector Mechanisms of an IgG1 and IgA1

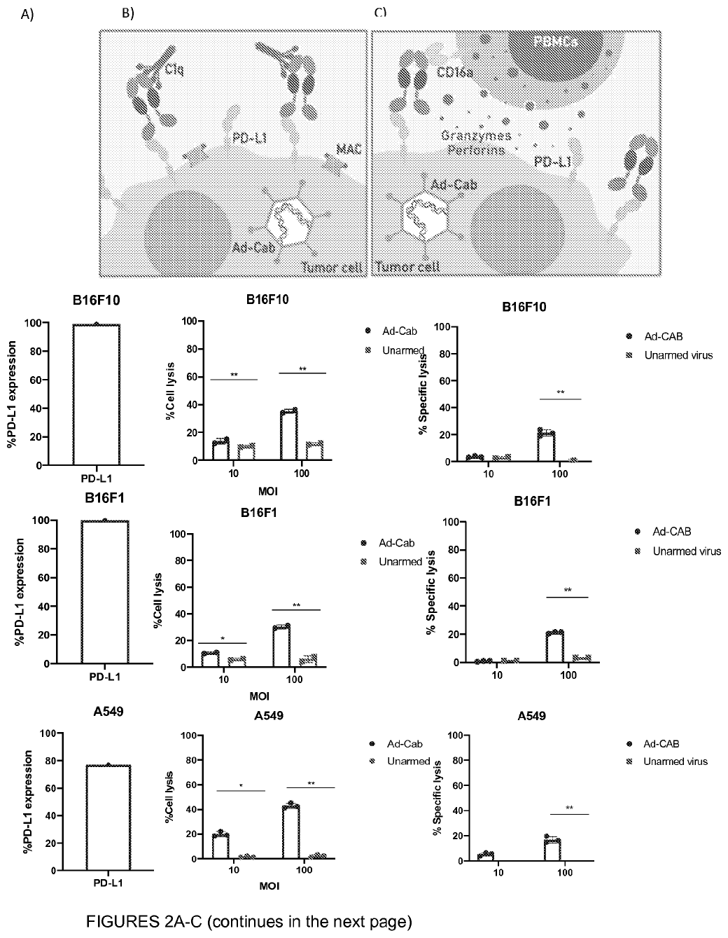

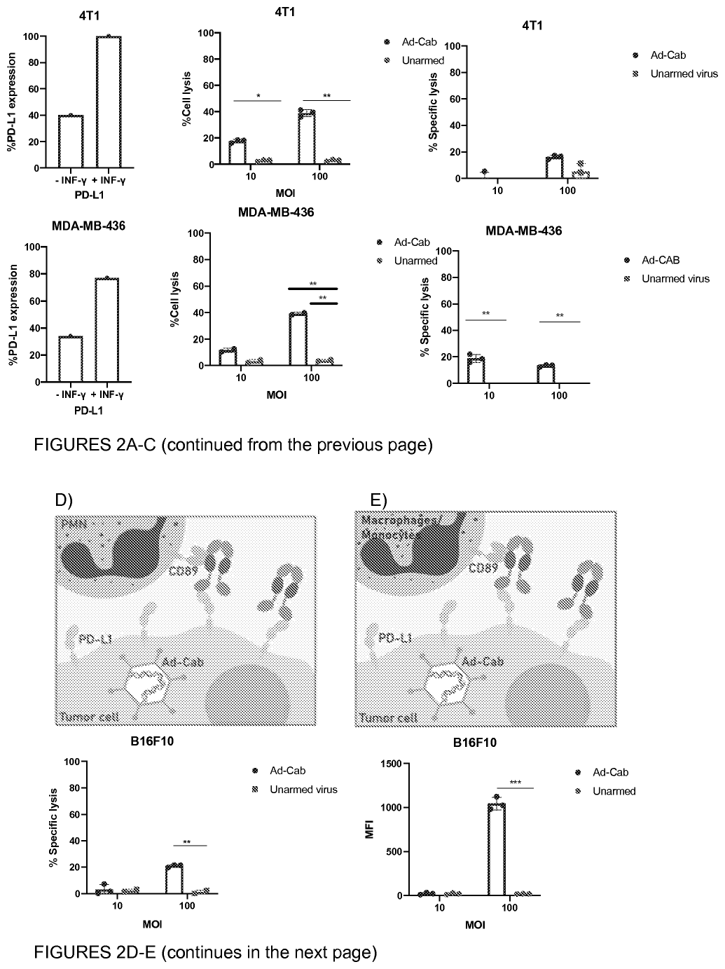

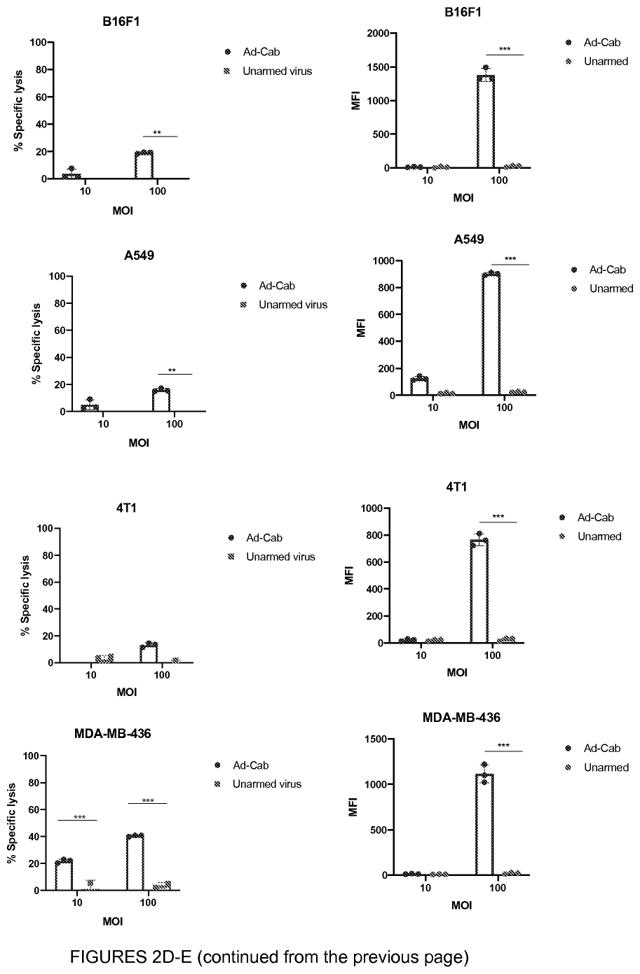

After testing expression and binding, we examined the ability of the Fc-fusion peptides to activate antibody effector mechanisms. Since the Fc entails a hybrid of an IgG1 and IgA1, CDC and ADCC were tested with both polymorphonuclear (PMN) and PBMCs on five different human and murine tumor cells lines expressing varying levels of PD-L1 (FIG. 2.a). Murine breast cancer (4T1) and melanoma cell lines (B16F10 and B16F1) were used since the oncolytic adenoviruses cannot induce oncolysis and cytotoxicity can then be attributed to the Fc-activation of effector mechanisms. For the CDC assay, cells were first infected at two different multiplicities of infection (MOIs), 10 and 100, and incubated for 48 hours to limit viral oncolysis and to secrete adequate levels of the Fc-fusion peptide. When the complement active serum was added, cell lysis could be observed with Ad-Cab infected cells (FIG. 2b). Already at MOI 10 cell lysis was occurring and was further augmented as the MOI increased to 100 in all five cell lines. As expected, cell lysis was not shown with the control virus, (unarmed oncolytic adenovirus Ad5/3-delta 24) in all conditions, further attributing cell death to CDC induction, especially in the human cell lines where viral oncolysis can be induced.

ADCC assays were then tested with two different immune populations; PBMCs (FIG. 2c) and PMNs (FIG. 2d). The same setup for the CDC assays were performed but instead of adding serum, PBMCs or PMNs isolated from buffy coats were used. Cell killing was determined by measuring the amount of lactate dehydrogenase (LDH) released from infected cells. In contrast to CDC, at MOI 10 minimal or no induction of ADCC could be seen with all cell lines infected with Ad-Cab when PBMCs or PMNs were added. Nevertheless, when the MOI increased to 100 cell lysis was observed with both populations. Interestingly, both PMNs and PBMCs were able to elicit similar levels of cytotoxicity with all the cells.

Finally, we wanted to test the ability of Ad-Cab to activate macrophages and induce ADCP. Using a similar setup as the previous experiments, we infected cells with Ad-Cab and unarmed virus at two MOI, 10 and 100. After 48 hours, cells were stained with carboxyfluorescein succinimidyl ester (CFSE) and macrophages were added at a ratio of 5:1 for 4 hours. The ability to elicit ADCP was then determined by the uptake of CFSE by macrophages. At MOI 10 no uptake of CFSE was observed with any condition yet at MOI 100, an increase of CFSE uptake by the macrophages can be observed in all cell lines when Ad-Cab was added. (FIG. 2e). Overall, the data demonstrates that the secreted Fc-fusion peptide is able to induce the effector mechanisms of both an IgG1 and IgA.

### Trogocytosis Drives the PMNs Mediated ADCC

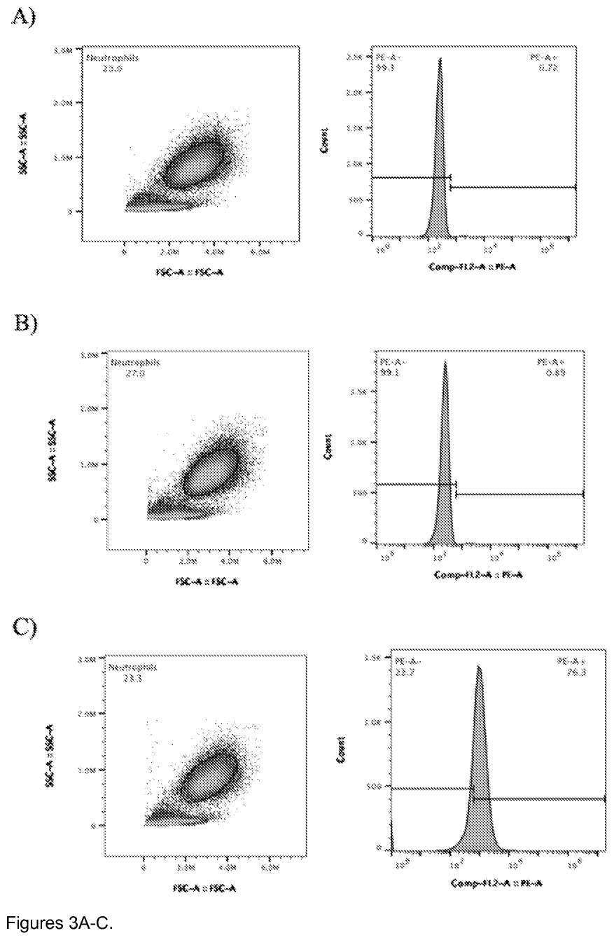

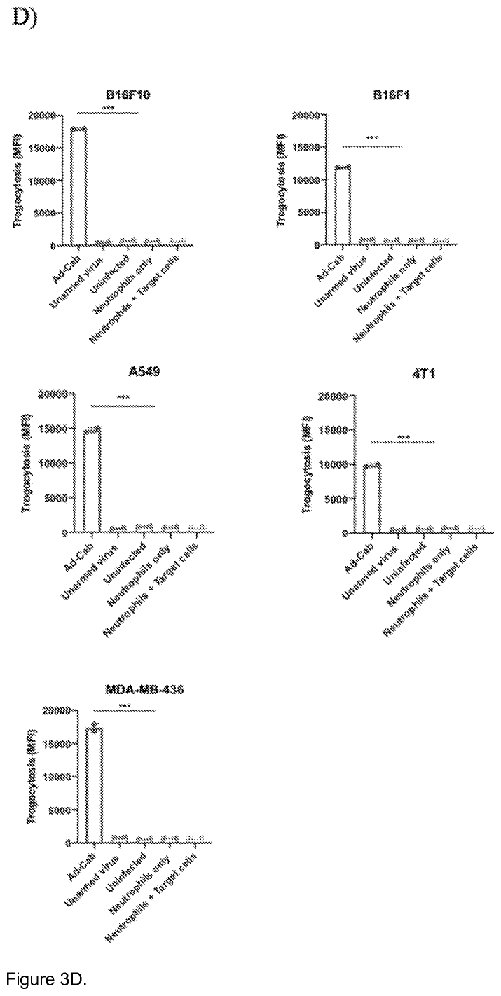

It has been previously shown that in order to initiate ADCC in vitro, PMNs adhere to the target cells establishing an immunological synapse with the antibody-opsonized tumor cells (Matlung, H. L. et al. 2018, Cell Rep. 23, 3946-3959.e6). This subsequently causes the disruption of their plasma membrane and the endocytosis of cytoplasmic fragments, leading to a necrotic type of cell death termed trogocytosis. In order to explore the possible nature of the cytotoxic mechanism during PMN based ADCC, we quantified the transfer of membrane from tumor cells to PMNs by flow cytometry. Virally infected cells had their lipid membrane labeled with 3,30-dioctadecyloxacarbocyanine perchlorate (DiO), a hydrophobic fluorescent dye, after which unstained neutrophils were added to the cell culture. Neutrophils were first gated using the side and forward scatter. The mean fluorescent intensity (MFI) of DiO was then measured on these neutrophils after incubation in three different conditions: without exposure to the target cells (FIG. 3a), with exposure to uninfected stained cells (FIG. 3b) and with exposure to Ad-Cab infected stained cells (FIG. 3c). When neutrophils were examined on their own, without former exposure to the target cells or when they were added to uninfected stained tumor cells, there was no DiO measured and no membrane transfer had happened. Yet, when the neutrophils were added to Ad-Cab infected cells, an uptake of DiO was observed by PMNs. This implies the uptake of the lipid membrane of the infected tumor cells from the neutrophils, which is a characteristic of trogocytosis. The same procedure was performed using all five cell lines and an increase in DiO MFI in the neutrophils was noted only when neutrophils had previously been exposed to cancer cells infected with the Ad-Cab and not with the other controls used (FIG. 3d). Hence, these findings add evidence in support that one of the mechanisms in which PMNs employ ADCC is trogocytosis.

### The Activation of Multiple Branches of the Immune System Induces Higher Cytotoxicity

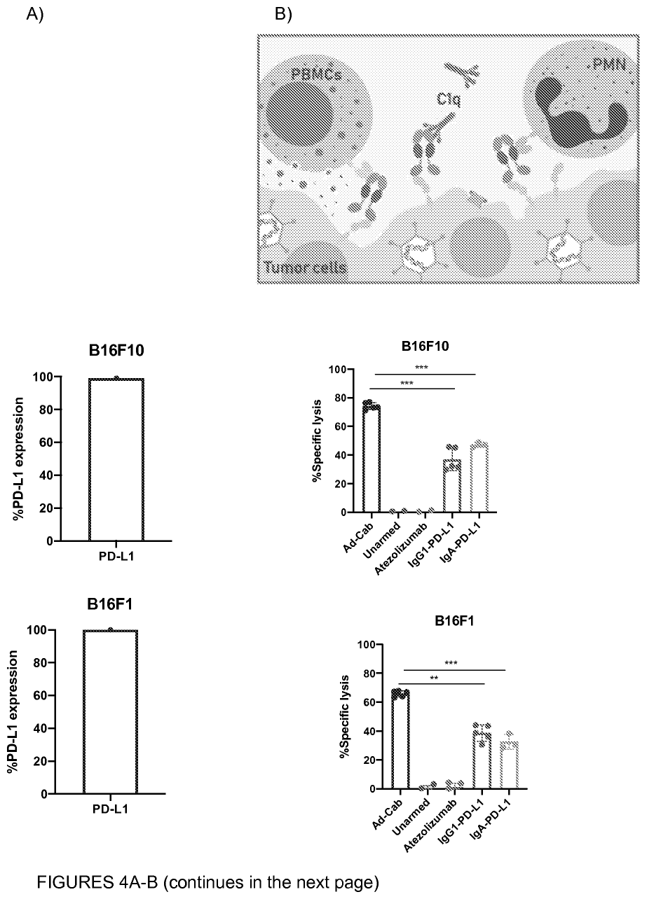

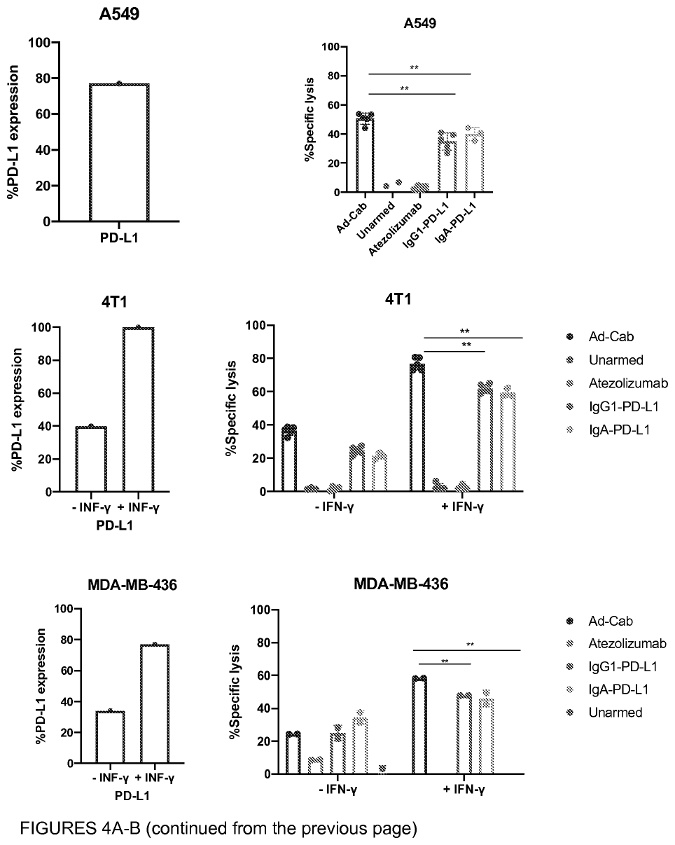

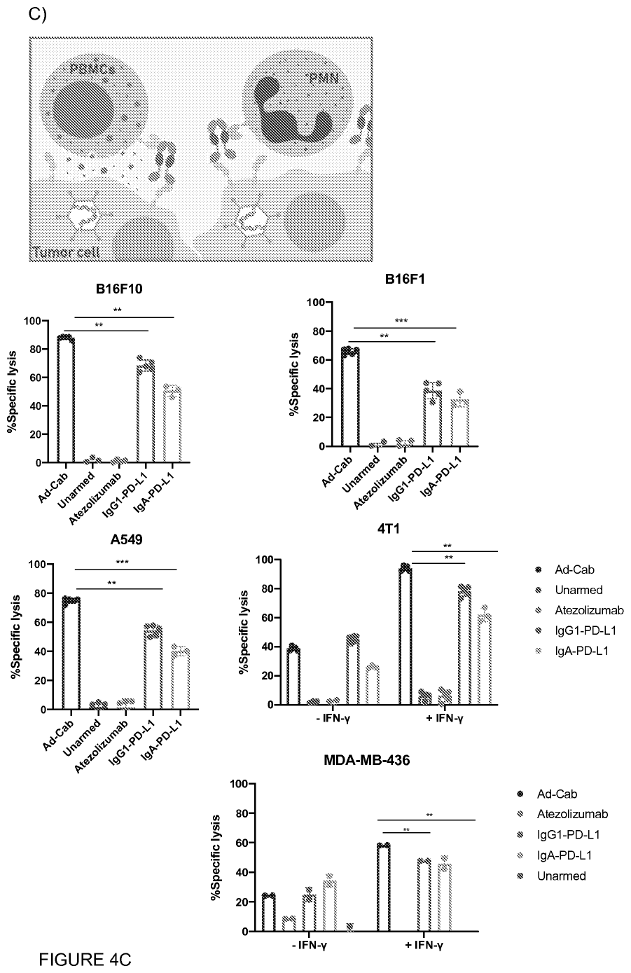

We hypothesized that a synchronous activation of the multiple branches of the immune system would lead to enhanced tumor cell killing and complete clearance of the tumor. To test this, we again performed the ADCC assays with different combinations of immune components such as PBMCs+PMNs or PBMCs+PMNS+serum with the same cell lines as previously expressing PD-L1 (FIG. 4a). Also, to further examine this we used Atezolizumab, which holds a N298A mutation abrogating its effector mechanisms, Atezolizumab without the mutation, designated IgG1-PD-L1, able to elicit effector mechanisms of an IgG1 and an IgA-PD-L1. When each immune component was added individually, Atezolizumab carrying the N298A mutation was not able to induce cell lysis. Interestingly, the functional IgG1 PD-L1 antibody was able to induce similar cell lysis levels as the Fc-fusion peptides when the complement system or PBMCs were added. Nevertheless, the IgG1 PD-L1 was able to induce only minimal cell lysis with PMNs compared to the Fc-fusion peptides. As expected, IgA-PD-L1 was only able to activate PMNs and not PBMCs or the complement system.

When PBMCs and PMNs were added together a significant cytotoxicity augmentation, compared to the cell populations alone, was observed with Ad-Cab (FIG. 4b). This was not seen with IgG1- or IgA-PD-L1 where cell lysis levels remained similar to when PBMCs or PMNs were added alone, respectively. Meanwhile, when all three components (fresh human serum, PBMCs and PMNs) were added together, enhanced cytotoxicity could again be noticed with Ad-Cab and IgG1-PD L1 (FIG. 4c). Interestingly, IgG1 PD-L1 showed a significant increase in cell lysis compared to when serum or PBMCs were added together. This again further reinforced the added benefit of activating multiple immune branches. Moreover, the addition of serum to the combination of PBMCs+PMNs also significantly increased cell lysis with Ad-Cab. This increase in cell lysis almost reached full clearance of PD-L1 expressing cells. Notably, this synergy effect was demonstrated with B16F10, B16F1 and A549 cells but not with 4T1 and MDA-MB-436 cells. We speculated that this was because most of the PD-L1 expressing 4T1 and MDA-MB-436 cells had already been eliminated when each component of the immune system was added individually. To examine this, PD-L1 expression on 4T1 and MDA-MB-436 cells was further increased by treating cells overnight with IFN-gamma before setting the ADCC assays (Garcia-Diaz, A. et al. 2017, Cell Rep. 19, 1189-1201). As anticipated, a higher tumor killing can be observed with all immune components added individually or in combination with INF-gamma treated 4T1 and MDA-MB-436 cells. Similar to the other cell lines, with the IFN-gamma treated cells, Ad-Cab was able to activate PMNs and induce higher killing with both combinations (PBMC+PMN and PBMCs+PMNs+serum) compared to IgG1 PD-L1.

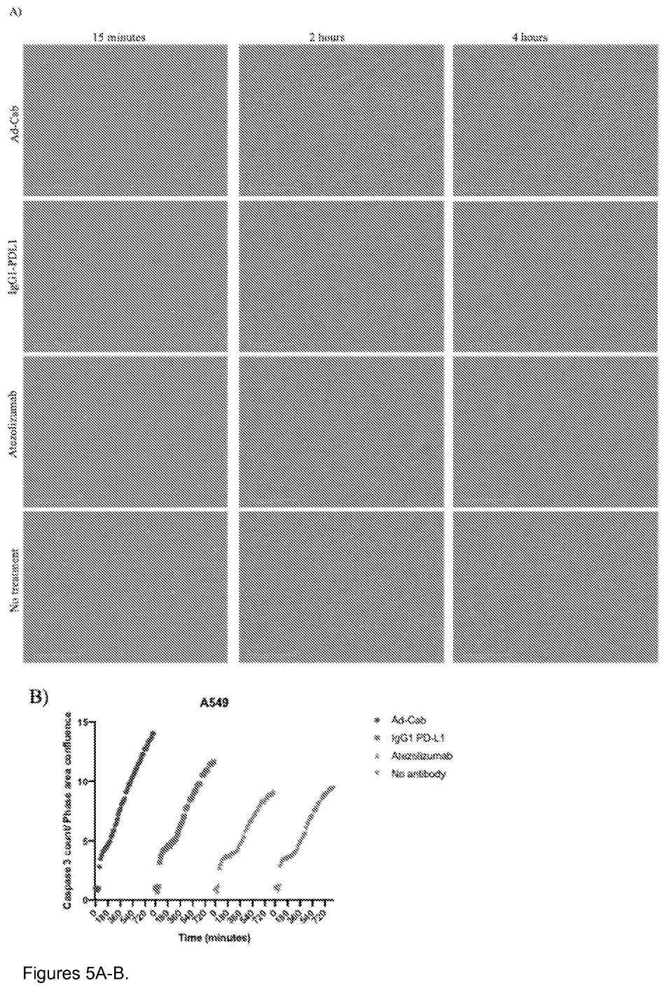

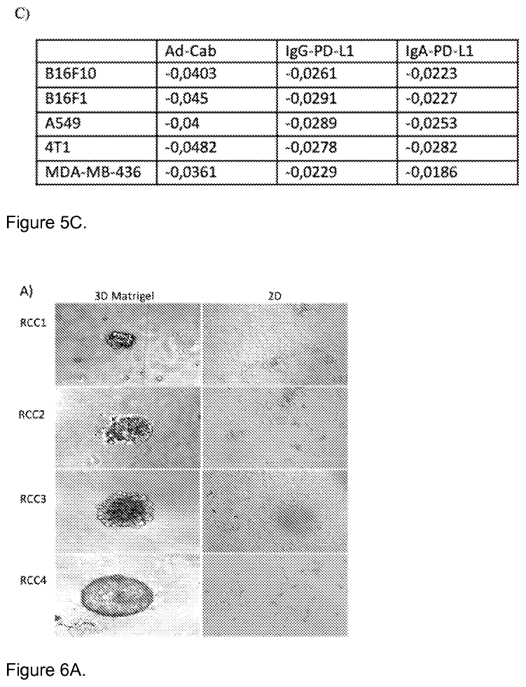

To further verify such data, we performed both live-cell microscopy and impedance-based real-time quantitative analysis (XCELLigence) to track target cells treated with purified Fc-fusion peptide (Ad-Cab) or therapeutic antibodies (Atezolizumab and IgG1-PDL1) following the addition of PBMCs and PMNs. With live-cell microscopy, A549 cells were monitored for 12 hours and death was determined using a Caspase-3/7 Green Reagent and phase confluency. We tested a range of effector:target (E:T) ratios with PBMCs and PMNs to evaluate ADCC. When we added PBMCs and PMNs at 100:1 and 40:1 (E:T) ratio, respectively, immune effector cells were very abundant and blocked visual representation of cell death events (data not shown). As for when PBMCs and PMNs were added at 1:1 and 0.4:1 E:T ratios, no cell death occurred with any treatment (data not shown). At E:T ratios of 10:1 and 4:1, PBMCs and PMNs respectively, live-cell imaging supported the LDH release data since apoptosis was observed when IgG1-PDL1 and Ad-Cab were added (FIG. 5a). Moreover, cell death was further enhanced with Ad-Cab compared to IgG1-PDL1 (FIG. 5b). Using XCELLigence, we analyzed cell-killing in real time and calculated the rate of cell death for each therapeutic antibody (IgG1- or IgA-PD-L1) and purified Fc-fusion peptide (Ad-Cab). The purified Fc-fusion peptide (Ad-Cab) had the highest killing rate in all cell lines, ranging from 0.0361-0.0482, compared to IgG-PD-L1 (0.0221-0.0289) and IgA-PD-L1 (−0.0186-0.0282) (FIG. 5c). Hence, we demonstrated that the Fc-fusion peptides augment immune-mediated apoptosis compared to IgG1-PD-L1, IgA-PD-L1 and the clinically used Atezolizumab in real time analysis.

### Characterization of Patient Derived RCC Organoids as Testing Platforms for Ad-Cab

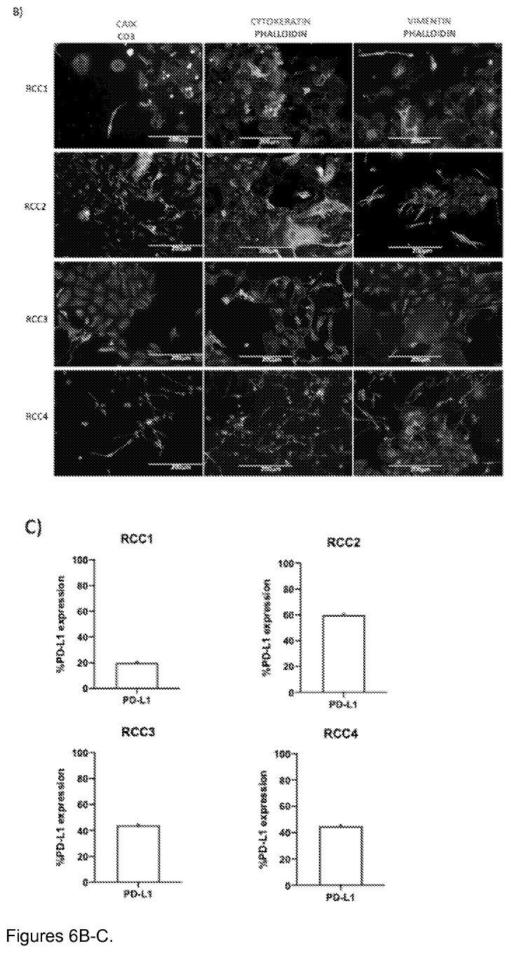

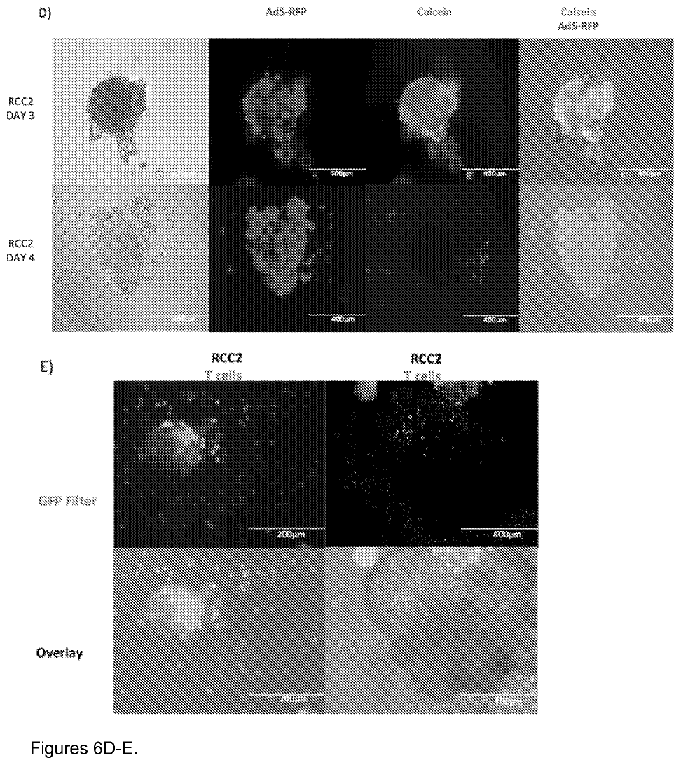

To further study the efficiency of Ad-Cab, we developed a novel testing platform using renal cell carcinoma (RCC) patients' derived organoids (PDOs). PDOs are three dimensional cultures that emulate the original complex tissue architecture and have been shown to be excellent screening platforms for individualized therapies. Freshly dissociated tumor tissue from four patients (RCC1-4) undergoing radical nephrectomies were obtained. Samples were grown either as 2D in 3D by embedding cells in Matrigel (FIG. 6a). A difference in self-organization can be observed where cells embedded in Matrigel formed 3D structures consisting of spheres. In order to assess the heterogeneity of the PDOs and to compare them to the corresponding tumor tissue that they originated from, we stained PDO cells allowed to grow as 2D on plastic with three commonly used stains to differentiate RCC (CAIX, Vimentin and Cytokeratin) and with an F-actin stain (Phalloidin) (FIG. 6b). CAIX is a marker of low endogenous oxygen level while vimentin is a type III intermediate filament protein. Both are highly sensitive and specific for clear cell renal cell carcinoma (ccRCC) (Yu, W. et al. 2017, BMC Cancer 17, 1-8). This is consistent with the staining since RCC2, RCC3 and RCC4 samples were shown to be positive for CAIX and vimentin and were characterized as ccRCC at the time of diagnosis. Surprisingly, RCC1 was both CAIX and vimentin positive despite being classified as a chromophobe RCC, a subtype that usually is not CAIX or vimentin positive. Yet, RCC1 had a focal expression of CAIX and lower expression of vimentin compared to the other samples, where staining was more diffused. The immunofluorescence analysis revealed that all four patient derived organoids consisted of renal cancer cells (FIG. 6b). Interestingly, non-cancerous cells were also shown in the PDOs due to the presence of cells that were only phalloidin positive. PD-L1 expression was then tested by dissociating the PDOs into single cells and evaluating expression using flow cytometry (FIG. 6c). Varying levels of expression of CD3−/PD-L1+positive cell was shown from the samples, from 20% to 66%. The data indicates that the organoids consist of a heterogeneous population of cells, mimicking in vivo tumor growth, and express PD-L1.

Subsequently, we tested whether oncolytic adenoviruses had the ability to pass through the Matrigel and infect the organoids. To this end we infected PDOs with an oncolytic virus expressing the red fluorescent protein (Ad5-Δ 24-RFP) to visualize the infection and the replication of the virus (FIG. 6d). The virus was added on top of the supernatant of the PDO cultures and after one day PDOs were already infected and expressing RFP. Expression kept increasing until reaching a maximum on day 3. To confirm whether the virus could induce oncolysis, a viability cell stain, Calcein AM, was added and monitored (FIG. 6d). Oncolysis was observed to start at day 3 with minimal death occurring, and by day 4 most cells were shown to be dead. To finalize the testing platform, we tested whether isolated PBMCs could travel through the Matrigel and surround the organoids. Before their addition to the organoid cultures, PBMCs were labelled with Calcine green and then added on top of the media (FIG. 6e). Within hours they could be seen to pass through the Matrigel and surround organoids. Hence, we demonstrate that the RCC PDOs can be used as testing platforms for the Ad-Cab viruses, since they express PD-L1, can be infected by oncolytic adenoviruses and infiltrated by PBMCs.

### Enhanced Efficacy of Ad-Cab in Patient Derived RCC Organoids

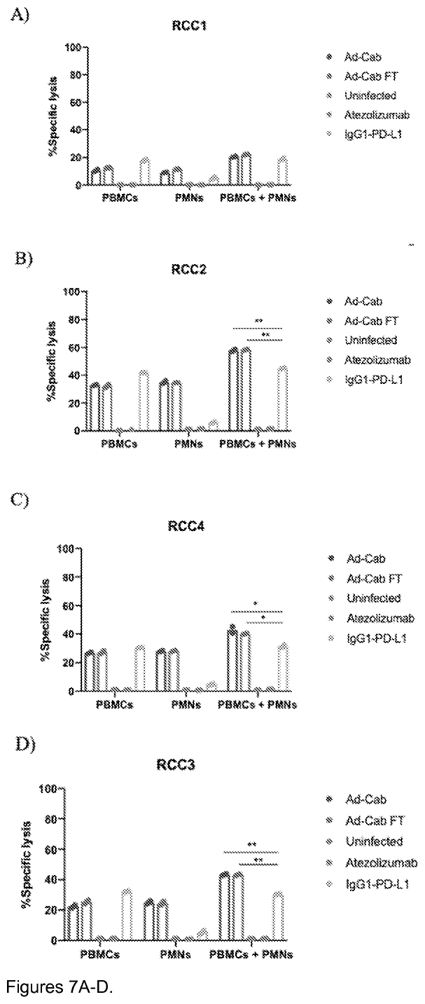

After optimizing the RCC organoids as a functional testing platform for the Ad Cabs, we utilized them to perform the ADCC experiments with PBMCs and PMNs (FIG. 7a-d). RCC organoids were first infected with the viruses or treated with the antibodies and incubated for three days. As evident from the ADCC results, when PBMCs were added to the organoids similar levels of cytotoxicity was observed with the Ad-Cab and the IgG1 PD-L1 antibody. Consistent with in vitro data, cytotoxicity could only be observed with the Ad-Cab and not with the IgG1 PD-L1 antibody when PMNs were added as the effector cells (FIG. 7a-d). When both populations of effector cells were added there was an enhanced killing with Ad-Cab compared to when each population was added individually (FIG. 7a-d), with all samples except RCC1 (FIG. 7a). This could be explained by the fact that all PD-L1 expressing cells were killed when each effector population was added individually. The added benefit of activating an additional effector population with Ad-Cab was evident since the killing efficiency in RCC2, 3 and 4 patient samples were greater when PBMCs and PMNs were added together. Thus, the PDOs further reinforced the efficacy of Ad-Cab and the significance of the synergism of the immune system for tumor eradication.

### Example 2

### Materials and Methods

### Cell Lines

All cell lines were purchased from the American Type Culture Collection (ATCC) and cultured in appropriate medium at 37° C. and 5% CO₂. Cell lines used in this study are human lung cancer A549, human triple negative breast cancer MDA-MB-436, murine triple negative breast cancer 4T1 and murine skin cancer B16K1 and B16F10. All cell lines were cultured until reaching passage 15 and routinely checked for mycoplasma infection.

### Virus Transgene Modifications

Adenoviruses were made conditionally replicating by using previously described protocols (Kanerva, A. et al. 2003, Mol. Ther. 8, 449-458). All adenoviruses contained a 24 base pair deletion in the E1A region and are of the serotype 5 but with a fiber of serotype 3 (Ad-5/3). Fc-fusion peptides were added to the adenovirus genome using previously described protocols (Hamdan, F. et al., Mol. Ther.—Methods Clin. Dev. 20 (2021) 625-634. In short, the gp19K+7.1k region was substituted with the Fc-Fusion peptides using Gibson-Assembly. Moreover, the Fc-fusion peptides were under a CMV promoter.

Amino acid sequences of the wild type and modified Fc-fusion peptides are shown in FIG. 8 and SEQ ID Nos:1-5.

### Cell Viability Assays

Cell viability was assessed by plating 10,000 cells and infecting them with various MOIs for three days. Death was then assessed by MTS according to the manufacturers protocol (Cell Titer 96 AQueous One Solution Cell Proliferation Assay; Promega, Nacka, Sweden). Spectrophotometric data was read using the Varioskan LUXMultimode Reader (Thermo Scientific, Carlsbad, C, USA).

### Serum Collection

Ten volunteers had 40 ml of their blood taken in BD Vacutainer collection tubes (BD Bioscience) and left to clot for 30 minutes at room temperature. Following clotting, blood samples were centrifuged for 5 minutes at 2500 rpm. Serum was then collected and frozen at −80° C. until further use.

### PBMC, PMN and Monocyte Collection

PBMCs and PMNs were separated and isolated from buffy coats as previously described (Cui, C. et al., STAR Protoc.2 (2021), 100845). Cells were cultured in 1×RPMI Roswell Park Memorial Institute (Gibco, Cat #21875034). From PBMCs, monocytes were collected as previously described (Evers, M. et al., Novel chimerized IgA CD20 antibodies: Improving neutrophil activation against CD20-positive malignancies, MAbs. 12 (2020).

### Mixed Leukocyte Reaction

Monocytes were differentiated into dendritic cells by culturing for seven days in DMEM low glucose supplemented with 10% FBS, 500U/ml of IL-4 (PeproTech, #200-04) and 250U/ml (Abcam, ab88382). After differentiation, PBMCs from a different donor were labeled with CFSE (Thermofischer), according to manufacturer's protocol, and co-cultured at a 1:10 ratio and treated with 1 μg/ml of either Atezolizumab (Invivogen) or Fc-fusion peptide. After five days, supernatants were collected and CFSE was measured in gated CD3+ CD8+ T cells by flowcytometry.

### Complement-Dependent Cytotoxicity Assay

CDC assays were performed either with purified Fc-fusion peptides or viruses. For viruses, 100,000 cells were plated and infected at indicated MOIs for 48 hours. As for Fc-fusion peptides, different concentrations indicated were added and incubated for 30 minutes. After incubation, 15.5% of complement active serum was added for four hours. Cells were then stained with 7-AAD (eBioscience) and lysis was measured using flow cytometry.

### Antibody-Dependent Cell Cytotoxicity Assays

ADCC assays were performed using either purified Fc-fusion peptides or viruses. Similar to the CDC assays, 15,000 cells were infected with the indicated MOIs for 48 hours while for Fc-fusion peptides the indicated concentration was added for 30 minutes. After the incubation, effector cells were then added at a ratio of 100:1 and 40:1 (Effector:Target) for PBMCs and PMNs, respectively. After four hours of incubation at 37° C., cells lysis was measured by calculating the release of endogenous LDH using a commercial kit (CyQUANT LDH Cytotoxicity Assay, Cat #C20303). Specific percent cell lysis was calculated using the following formula: (“experimental LDH release”−“effector plus target spontaneous”)/(“target maximum”−“effector plus target spontaneous”)×100. Experimental LDH release corresponds to the signal measured by the treated samples, effector plus target spontaneous corresponds to the release of LDH when effectors and targets are incubated, and target maximum corresponds to when target cells are treated with cell lysis buffer.

### Whole Blood Assay

Whole blood was collected from three healthy volunteers in BD Vacutainer Heparin plasma tubes (BD biosciences). 200 μl of unmanipulated blood was then incubated with 20 μg/ml of antibody or Fc-fusion peptides for 24 hours at 37° C. After 24 hours, samples were treated with ACK buffer to lyse red blood cells and then stained with CD3, CD15, CD14, CD56 and CD11c to differentiate immune populations. Counting beads were then added before performing flow cytometry to calculate absolute numbers (Biolegend, Cat #424902).

### Animal Experiments

For syngeneic mice experiments, BALB/c or C57BL/6 4-8-week-old immunocompetent mice, purchased from Envigo, were injected with 300,000 4T1 or 500,000 B16K1 cell in the right flank, respectively. After 9 days, tumors were palpable and then followed a treatment schedule of 4 treatments separate by two days of break in-between. Viruses or PBS were injected intratumorally at a final volume of 25 μl while antibodies were administered intraperitoneally at final volume of 100 μl. Viruses were administered at a concentration of 1×10⁸ viral particles per mouse while 100 μg of antibody was administered per mouse. Tumor size was calculated using the following formula: (long side)×(short side)²/2.

As for xenograft mice models, 4-6-week-old immunodeficient Nod.CB17-Prkdcscid/NCrCrl mice were purchased from Charles River. For tumor implantation, mice were injected with 5×10⁶ A549 cells subcutaneously in the right flank. On the same day, 5×10⁶ PBMCs extracted from the same donor were injected intraperitoneally for engraftment. After tumors were palpable, mice were given two doses of virus at a concentration of 1×10⁹ viral particles per mouse.

All animal experiments have been approved and reviewed by the Experimental

Animal Committee of the University of Helsinki and Provincial Government of Southern Finland (license number ESAVI/11895/2019).

### Biodistribution Analysis

After animals were sacrificed, tumors, livers and peripheral blood were collected for processing. Tumors and livers were passed through a 0.22 μm cell strainers to create single-cell suspension. Samples were then centrifuged for 10 minutes at 500g to pellet cells and collect the supernatant for further processing. While as for blood, samples were centrifuged for 30 minutes at 500g and serum was collected. Since the Fc-fusion peptides contain a C-terminal His-tag, a His-tag ELISA was used to determine concentrations from the supernatant and serum samples collected (Cell Biolabs, Cat #AKR-130).

### Flowcytometry Analysis

All flow cytometry samples were run either with the BD Accuri 6 plus (BD Bioscience) or Fortessa (BD Bioscience). Both human and murine samples had two antibody panels each that were used. Panel 1 includes FITC anti-mouse NK1.1 (Thermo Fisher Scientific Cat #11-5941-85, RRID:AB_465319), PE anti-mouse PD-1 (BioLegend Cat #135206, RRID:AB_1877231), PeCy7 anti-mouse CD4 (Thermo Fisher Scientific Cat #25-0041-82, RRID:AB_469576), PerCp/Cy5.5 antimouse CD107a (BioLegend Cat #121626, RRID:AB_2572055) and Pacific Blue anti-mouse CD3 (BioLegend Cat #100214, RRID:AB_493645). Panel two included APC anti-mouse Ly6C (BioLegend Cat #128015, RRID:AB_1732087), PE antiLy6G (BD Biosciences Cat #551461, RRID:AB_394208), PerCP Cy5.5 anti-mouse CD11b (Thermo Fisher Scientific Cat #45-0112-82, RRID:AB_953558), BV650 anti-mouse F4/80 (BD Biosciences Cat #743282, RRID:AB_2741400) and PECy7 anti-mouse CD11c Fisher Scientific (Thermo Cat #25-0114-829, RRID:AB_469590). For humans, the first panel included FITC anti-human CD56 (BioLegend Cat #304604, RRID:AB_314446), PerCP anti-human CD8alpha (BioLegend Cat #300922, RRID:AB_1575072), PE-Cy5 anti-human CD4 (Thermo Fisher Scientific Cat #15-0049-42, RRID:AB_1582251), PE-Cy7 anti-human CD3 (BioLegend Cat #300316, RRID:AB_314052), Pacific blue anti-human PD-1 (BioLegend Cat #329915, RRID:AB_1877194) and APC anti-human CD107a (BioLegend Cat #328620, RRID:AB_1279055). The second panel for human samples included PE-Cy7 antihuman CD3 (BioLegend Cat #300316, RRID:AB_314052), APC anti-human CD11c (BioLegend Cat #371505, RRID:AB_2616901), Pacific Blue anti-human CD15 (BioLegend Cat #323021, RRID:AB_2105361) and PE anti-human CD14 (BioLegend Cat #301805, RRID:AB_314187).

### Statistical Analysis

All statistical analysis were performed with GraphPad Prism 7 (GraphPad Software, La Jolla, California, USA). Statistical tests used were either unpaired t-test and Two-way ANOVAs with a post-hoc (Tukey's multiple comparison tests). All n≥3 and significance were set at *p<0.05, ** p<0.01, *** p<0.001 and **** p<0.0001. Error bars represent standard error of mean (SEM).

### Results

Cab-FT Activates Higher ADCC with PBMCs at Lower Concentrations than Cab

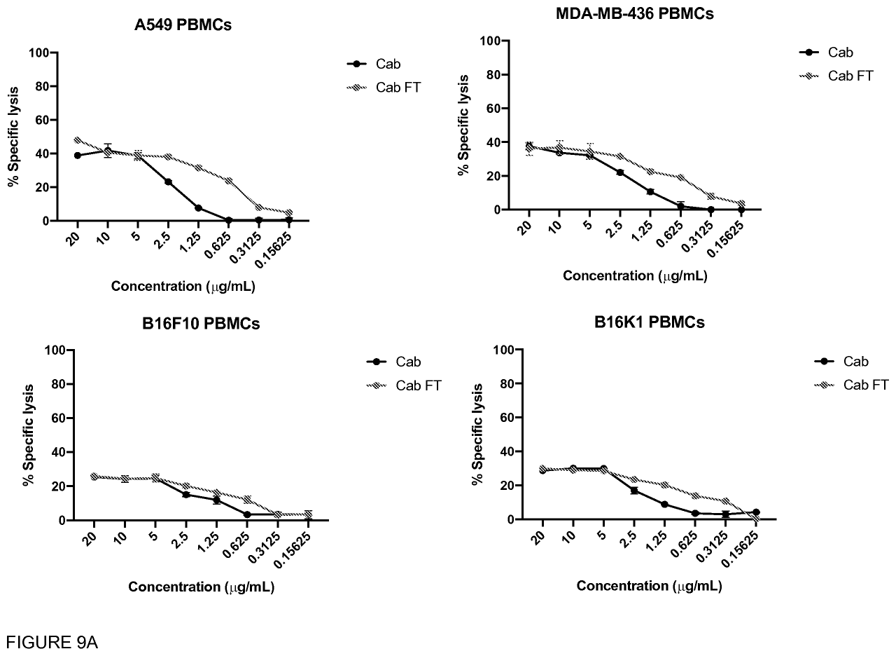

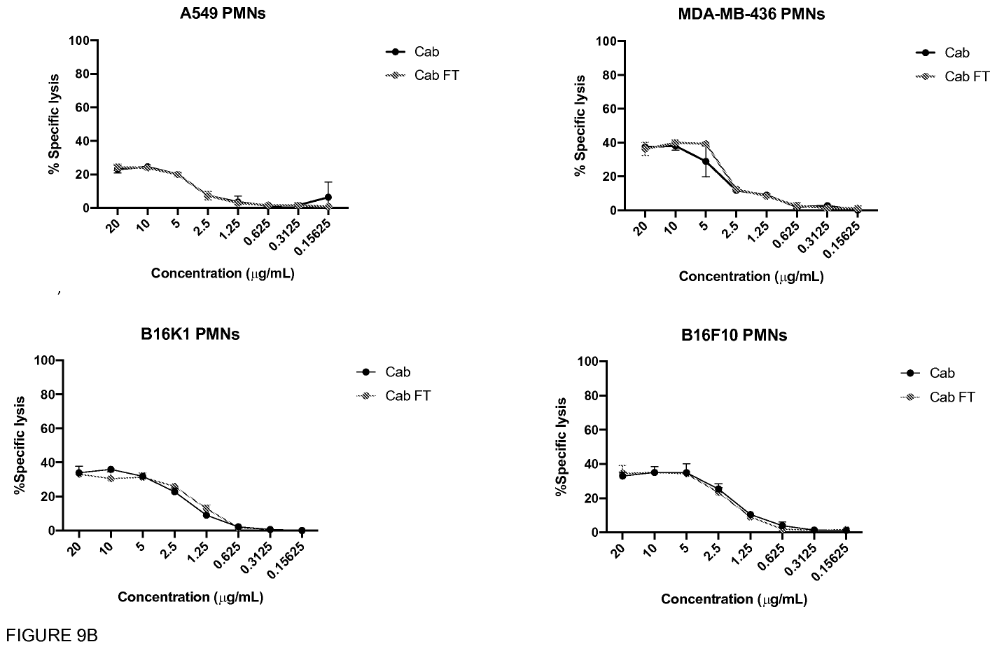

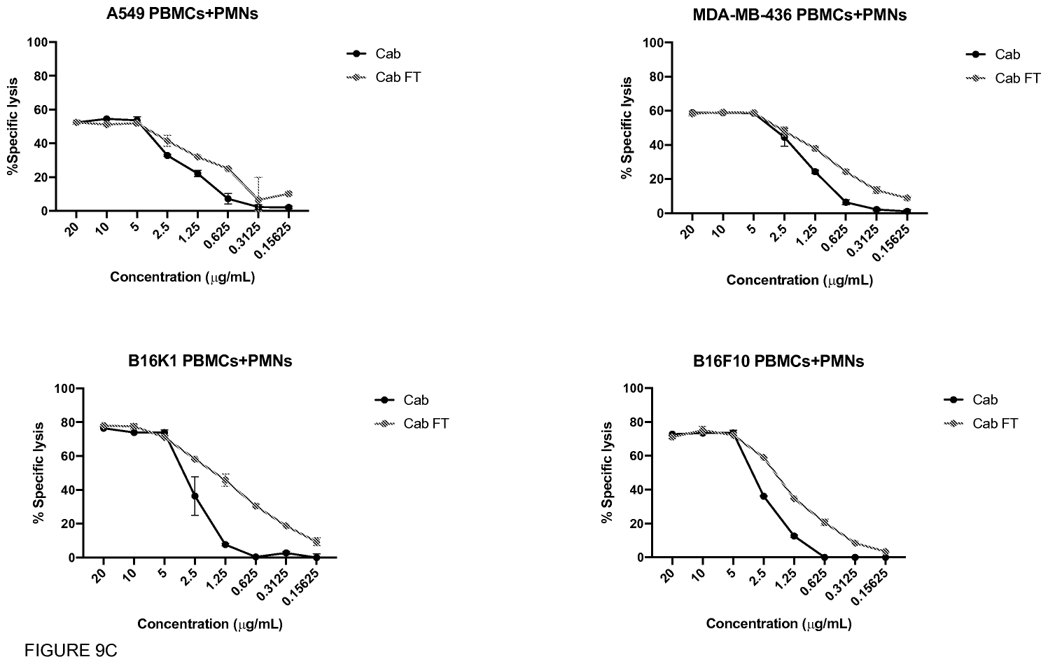

In example 1 we designed a novel Fc-fusion peptide (Cab) consisting of a PD1 ectodomain (binding to PD-L1) connected to a cross-hybrid IgGA Fc via a GGGS linker. Such Fc-fusion peptide was able to display effector mechanisms of both an IgG1 and IgA which increased tumor killing compared to clinically approved PD-L1 antibodies or with an IgG1 or IgA backbone alone. To further improve Cab, four point mutations (H268F/S324T/S239D/1332E) were added to the IgG1 portion of the cross-hybrid IgGA Fc (Cab FT) increasing its activation of NK cells (Moore, G. L. et al., MAbs. 2 (2010) 181). We performed ADCC experiments with isolated PBMCs and different concentrations (20 μg/ml-0.15625 μg/ml) of Cab and Cab FT. These experiments were performed with two murine (B16F10 and B16-K1) and human cell lines (A549 and MDA-MB-439) since the PD-1 ectodomain is able to bind to both murine and human PD-L1. With all cell lines, a clear trend can be observed in which Cab FT was more potent in lower concentration between 2.5 μg/ml-0.3125 μg/ml compared to Cab (FIG. 9A). Yet, at high concentrations of 20 μg/ml-5 μg/ml both Cab and Cab FT had a similar efficiency of tumor killing (FIG. 9A).

We then repeated the same experiments but with PMNs as an effector population. As expected, no difference in killing was observed between Cab FT and Cab since IgG1 sub-optimally activates neutrophils (FIG. 9B). Surprisingly, no increase in tumor killing was observed when serum was added even though the point mutations in Cab-FT were shown to increase C1q binding.

After seeing an increase activation of NK cells with Cab FT, we wanted to see if we could still observe such effect in more physiological conditions where both PBMCs and PMNs would be present. Similar to previous data, Cab FT was still superior to Cab at lower concentrations further emphasizing the added gain in tumor killing (FIG. 9C). Overall, the data demonstrate that the point mutations in Cab-FT increases ADCC killing with PBMCs at lower concentrations.

Cab and Cab-FT does not Induce Leukocyte Killing and Blocks the PD-1/PD-L1 Axis

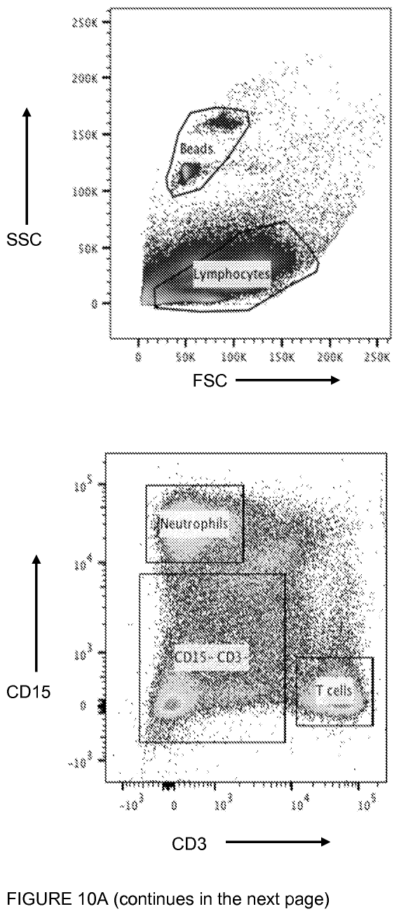

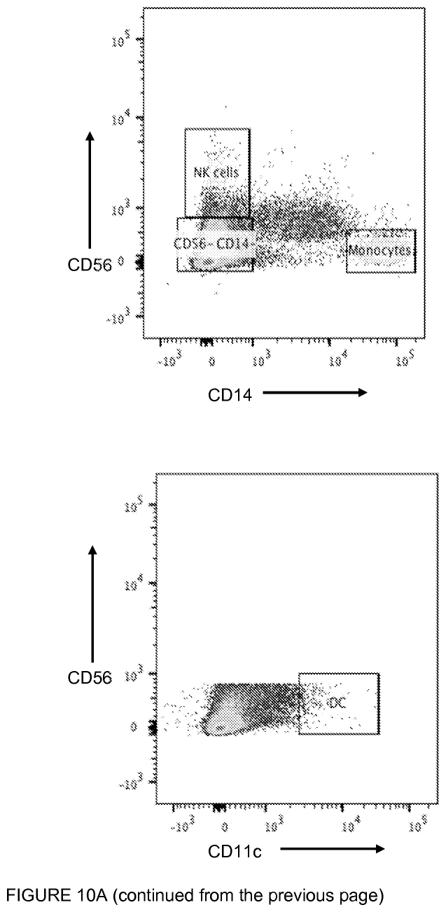

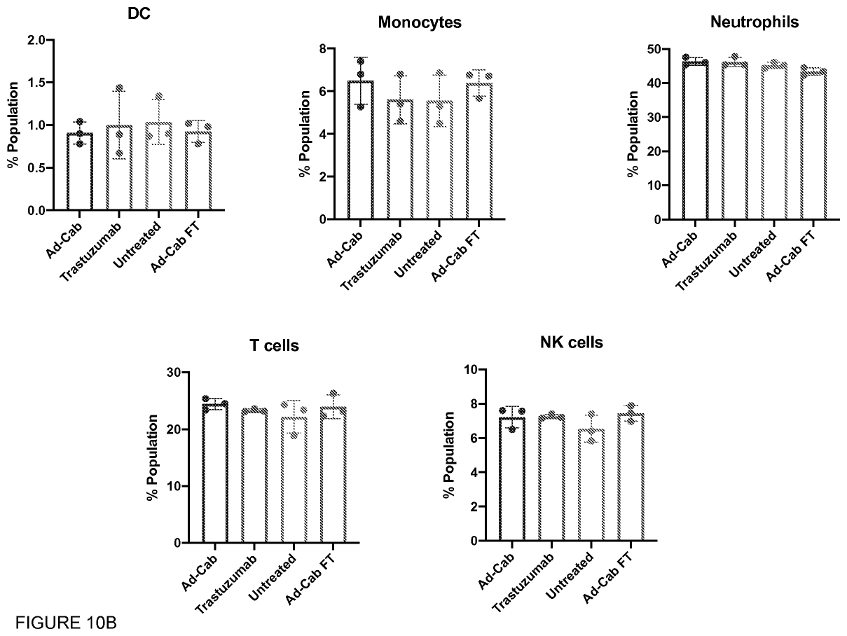

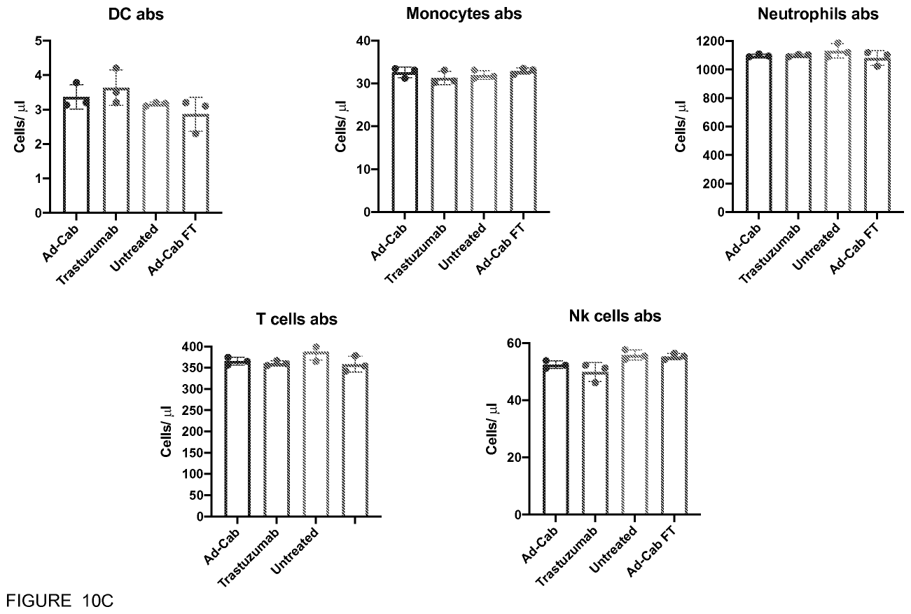

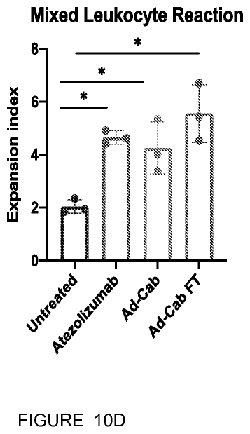

PD-L1 expression is not limited to tumor or healthy cells but can also be expressed by many immune cells such as macrophages, dendritic cells (DC) and monocytes. Yet, the copy number of PD-L1 per cell has been documented to be lower compared to tumor cells. This plays to our advantage since the copy number of a target epitope is an essential requirement for antibody effector mechanisms to be activated. Consequently, Cab and Cab FT could potentially not harm crucial immune cells. To test this, we performed a whole blood assay where unmanipulated blood from three different donors were incubated with 20 μg/ml of both Cab and Cab-FT. Under the presence of all effector populations, we analyzed if a decrease in cell percentage or absolute number was observed with T cells, NK cells, macrophages, DC and neutrophils (FIG. 10A). No differences in percentage (FIG. 10B) or absolute numbers (FIG. 10C) was observed among both Fc-fusion peptides and untreated samples or negative control Trastuzumab (binding to Her-2 and not found on immune cells) treated samples. This indicates that even though Cab and Cab FT induce high tumor killing it does not kill low PD-L1 expressing immune cells.

Certain studies have shown that increasing Fc effector mechanisms in PD-L1 checkpoint inhibitors can also lead to higher disruption of the PD-L1/PD1 axis. To test whether the enhanced killing of Ad-Cab FT was due to the point mutations and not to an increased PD-L1/PD1 disruption we performed a mixed leukocyte reaction. In this assay the PD-L1 and PD1 axis is analyzed by co-incubating monocytic-derived DCs from one donor with CFSE stained PBMCs from another donor. Due to the presence of PD-L1 and PD1 among both cells very little proliferation should be obvert, yet under a PD-L1 checkpoint inhibitor an increase expansion should be observed. Both Cab FT and Cab had a very similar expansion index to each other and to clinically approved PD-L1 checkpoint inhibitor, Atezolizumab (FIG. 10D). Hence, Cab and Cab FT inhibit in a similar level the PD1/PDL1 axis.

Ad-Cab and Ad-Cab FT have a Similar Oncolytic Fitness and Express the Respective Fc-Fusion Peptide

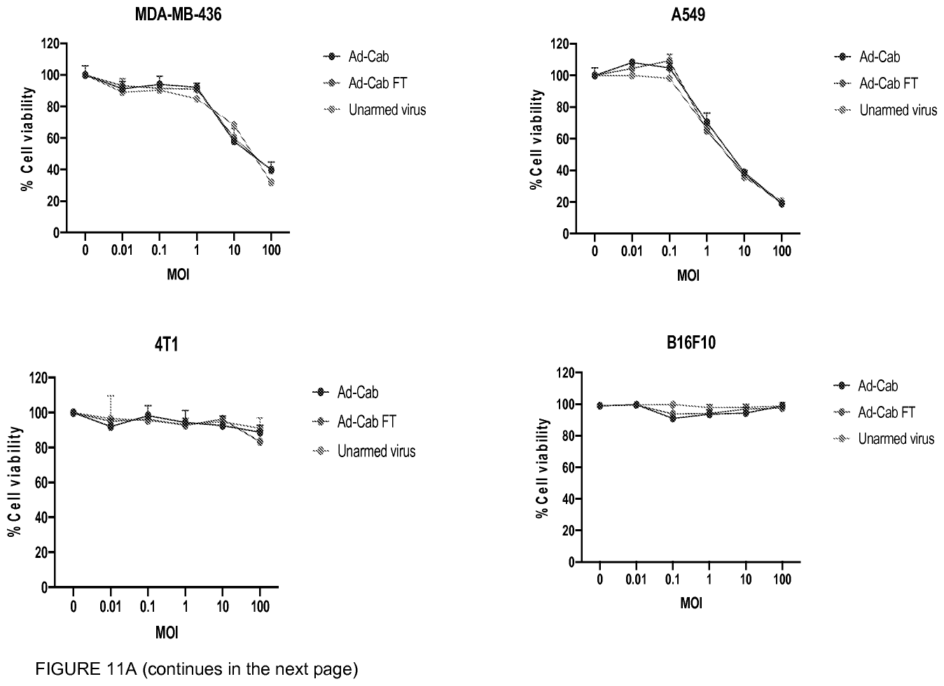

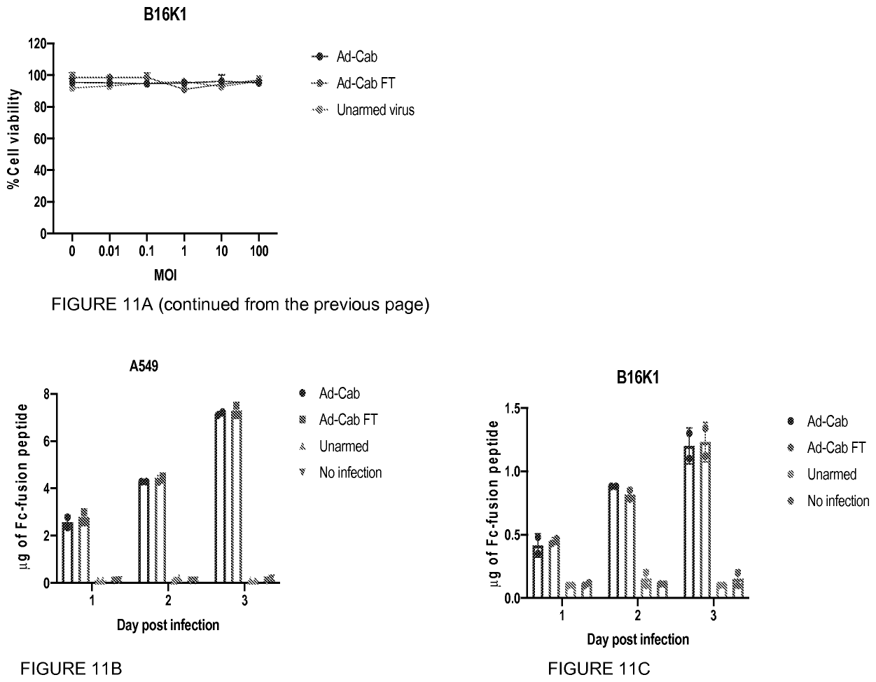

In order to circumvent safety concerns, Cab and Cab FT were cloned into an oncolytic Adenovirus-5/3 calling each virus Ad-Cab and Ad-Cab FT, respectively. After cloning and isolating the viruses, an MTS assay was performed to assess the oncolytic fitness. In human cell lines (MDA-MB-436 and A549) a clear cell lysis was observed as the MOI increased up to 100. Ad-Cab and Ad-Cab FT had a very similar level of cell lysis and also comparable to unarmed Ad-5/3 Δ24 (FIG. 11A). This indicated that both Ad-Cab and Ad-Cab FT had a similar oncolytic fitness, and that the gene manipulation did not affect fitness/oncolysis/functionality. As expected, no cell lysis was observed in any of the murine cell lines (B16F10, B16-K1 and 4T1) (FIG. 11A). This is due to the lower replication of Ad-5 viruses in murine cells. Moreover, Ad-Cab and Ad-Cab FT have the same oncolytic fitness as unarmed Ad-5/3 Δ24.

Subsequently, we tested whether Ad-Cab and Ad-Cab FT could express and also secrete the Fc-fusion peptides to the supernatant. Both Cab and Cab-FT had a His-tag allowing for quantification. When A549 cells were infected at 100 MOI a clear secretion of Fc-fusion peptide was observed and was increasing until reaching three days. Around 7 μg/ml of Fc-fusion peptides could be observed being released by day three (FIG. 11B). Similar results were also shown with B16-K1 yet with lower amounts being secreted due to the lower replication in murine settings (FIG. 11C). Such results indicate that Ad-Cab and Ad-Cab FT are able to express and secrete adequate levels of the Fc-fusion peptide in both human and murine settings.

Ad-Cab FT Induces Higher Tumor Killing at Lower Concentrations when PBMCs are Added

To test whether the secreted Fc-fusion peptides from the adenovirus were functional and could observe an added benefit with Cab-FT, we infected target cells with Ad-Cab and Ad-Cab FT and performed again ADCC assays. In the human settings, cells were infected at different MOIs ranging from 10-100. In accordance to our data, at lower MOIs Ad-Cab FT outperformed Ad-Cab when PBMCs were added as effector population. Moreover, no death was observed with Ad-5/3 Δ24 indicating that cell lysis was due to the Fc-fusion peptide and not because of the viral oncolysis. In the murine setting, higher MOIs were used since the secretion of the Fc-fusion peptide is lower compared to human cells. Similar to the human cell line data, a higher specific cell lysis could be observed with Ad-Cab FT at lower MOIs compared to Ad-Cab. We then repeated the same experiments with either adding PMNs or complement-active serum. As expected, when PMNs or serum was added a clear cell lysis was observed among Ad-Cab and Ad-Cab FT, and not Ad-5/3 Δ24, with similar specific cell lysis levels at different MOIs.

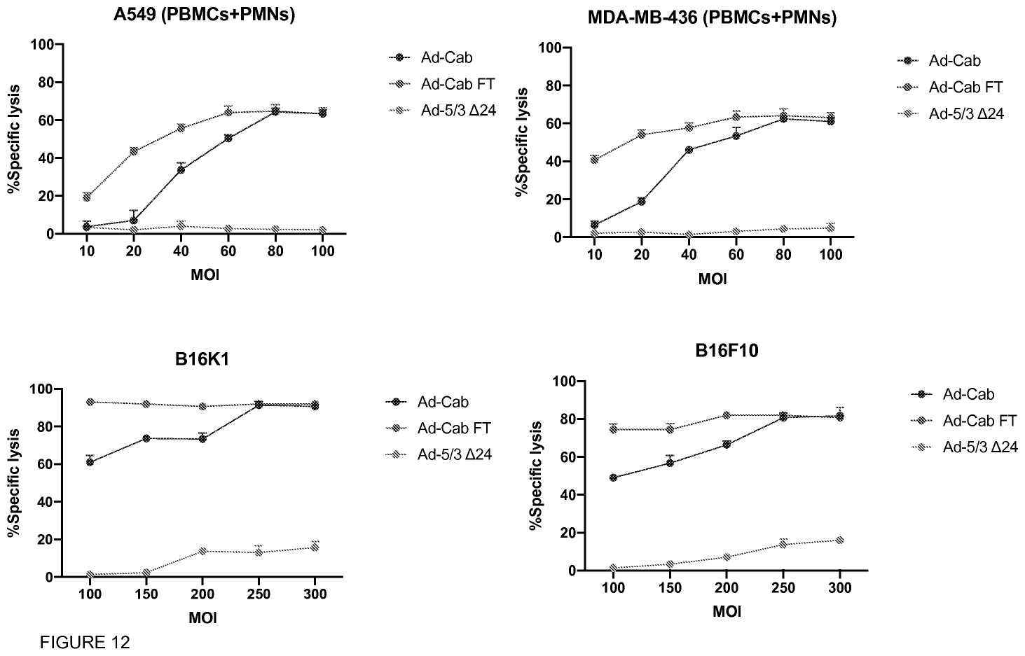

We then performed the same experiments but with the combinations of effector populations such as PBMCs+PMNs (FIG. 12). As expected, Ad-Cab FT was superior to Ad-Cab at lower concentrations. Therefore, Ad-Cab FT is able to secrete functional Cab FT and induce high tumor killing at lower MOIs.

Ad-Cab FT Induces Faster Killing than Ad-Cab

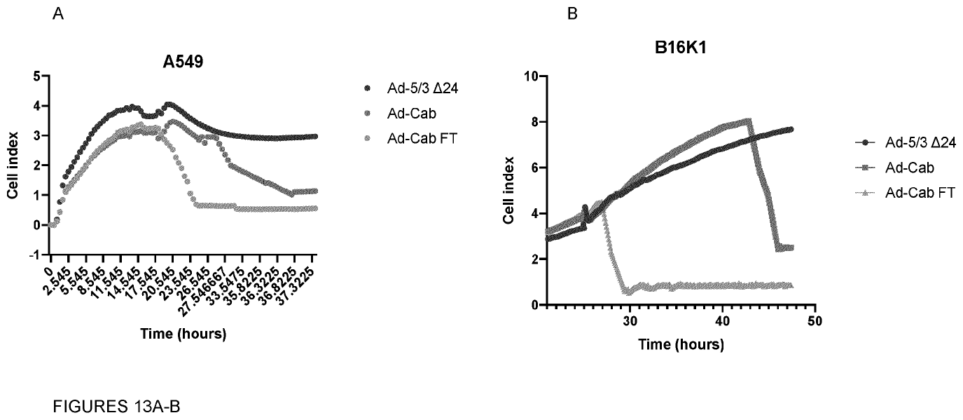

To further elude the advantages of Ad-Cab FT, we examined the kinetics of tumor killing using an impedance-based real-time quantitative analysis (XCELLigence). A549 cells were infected at 30 MOI while B16F10 were infected at 100 MOI and co-incubated with both PBMCs and PMNS (FIG. 13A). Cell killing was then analyzed in real time and with A549 at around 18 hours cell killing could be observed only with Ad-Cab FT. No death was recorded with either Ad-Cab or Ad-5/3 Δ24 at 18 hours. Ad-Cab started to record cell killing at around 32 hours while with Ad-5/3 Δ24 at around 40 hours. Moreover, within 24 hours Ad-Cab FT reached its final cell killing capacity, before viral oncolysis was recorded with Ad-5/3 Δ24, for which Ad-Cab it took more than 36 hours. The level of cell killing was superior with Ad-Cab FT compared to Ad-Cab which correspond with our previous data. In agreement with the A549 data, similar kinetics were also observed with B16K1 (FIG. 13B). Yet, cell-killing was observed later at around 27 hours for Ad-Cab FT while for Ad-Cab it was seen at around 42 hours. This data clearly indicates that Ad-Cab FT does not solely kill tumor cells at a higher efficiency but also at a faster pace than Ad-Cab.

Ad-Cab FT Control Tumor Growth In Vivo with B16-K1 and 4T1

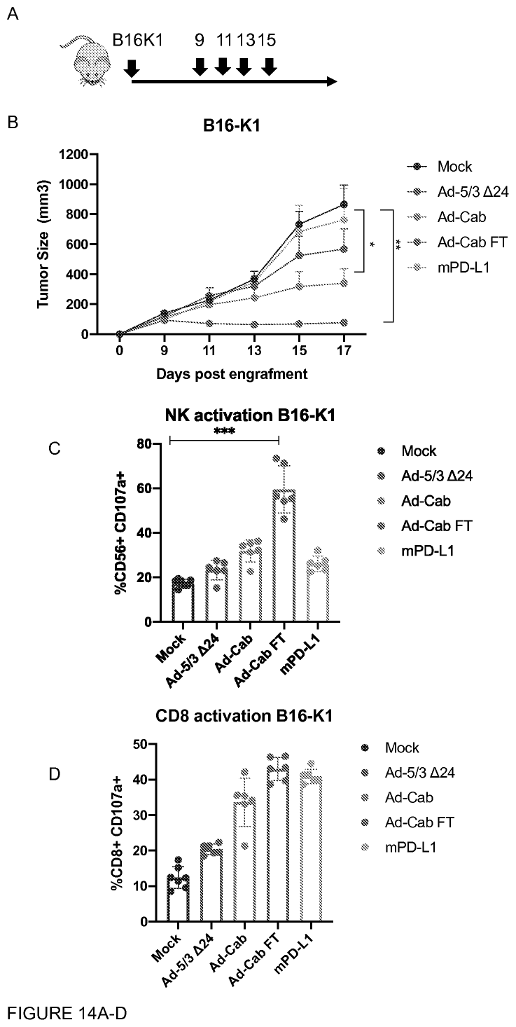

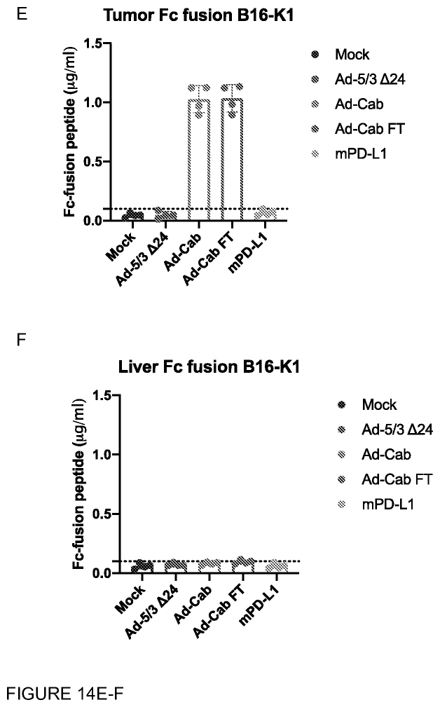

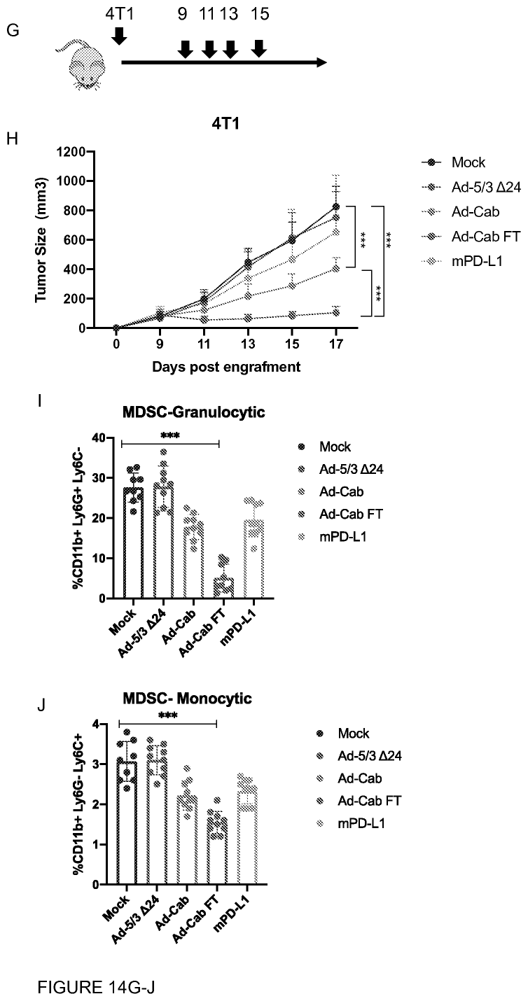

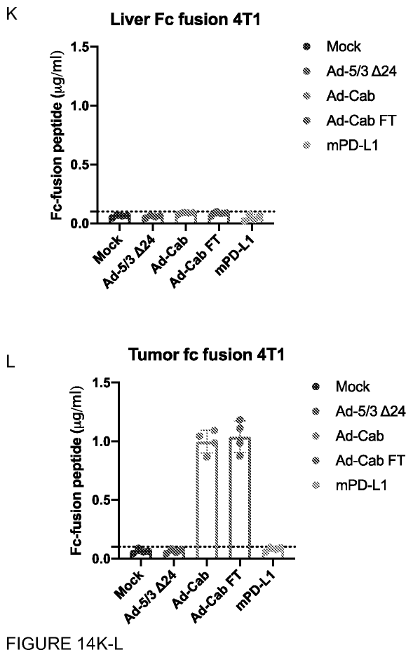

Following the in vitro data, we assessed the efficacy of Ad-Cab and Ad-Cab FT in vivo with different tumor mouse models. For the first tumor model we used B16-K1 due to its high expression of PD-L1. Mice were sub-divided into five treatment groups receiving different treatments: PBS (mock), Ad-5/3 Δ24, Ad-Cab, Ad-Cab FT and mPD-L1. Four injections of each treatment were given intratumorally (viruses or PBS) or intraperitoneally (antibodies) with a two day break in-between injections (FIG. 14A). Usually, 109 viral particles are administered per mouse for oncolytic adenoviruses therapy. Since Ad-Cab FT was shown to work at lower MOIs, mice are administered with one log lower dose, 108 viral particles per mouse, than usual. As expected, Ad-Cab FT outperformed all other treatment groups (FIG. 14B). After the last dose, mice were sacrificed two days later to investigate the tumor microenvironment. Interestingly, similar to in vitro, a higher NK cell activation was seen in the Ad-Cab FT explaining the better tumor control than Ad-Cab (FIG. 14C). A high upregulation of CD107a was observed with NK cells from the Ad-Cab FT group, which signifies a release of cytotoxic molecules such as perforins and granzymes. Like NK cells, an upregulation of CD107a was also seen with CD8+ T cells with groups Ad-Cab, Ad-Cab FT and mPD-L1 due to the PD1/PD-L1 inhibition (FIG. 14D). Analyzing the tumor microenvironment, a clear increase in NK cell infiltration is observed in Ad-Cab FT treated groups but similar levels of CD8+ T cells or CD4+ T cells can be seen in all groups. We then tested the biodistribution of the Fc-fusion peptide in the tumor and liver. Around 1 μg/ml could be observed in the tumor for Ad-Cab and Ad-Cab FT groups (FIG. 14E) while below detection levels could be seen in the liver (FIG. 14F). Overall, Ad-Cab FT was able to control tumor growth at lower dosages than Ad-Cab and have a safe biodistribution.

After observing the added benefit of Ad-Cab FT with B16-K1, we repeated the same experiment but with a highly immunosuppressive and fast-growing tumor model, 4T1. Similar groups, schedule and dosages were applied (FIG. 14G). Comparable to B16-K1, Ad-Cab FT groups had the best tumor control compared to other groups (FIG. 14H). Ad-Cab was also able to control tumor growth better than mPD-L1 or mock. After sacrificing the mice, we evaluated different immune cell populations. One of the main reasons 4T1 tumors are immunosuppressive is due to the high infiltration of myeloid derived suppressor cells (MDSC). A decrease in both MDSC-granulocytic (FIG. 141) and MDSC-monocytic (FIG. 14J) cells can be seen in Ad-Cab FT groups, most likely due to the high expression of PD-L1. Furthermore, an increase in NK cells (can be seen in Ad-Cab FT treated groups along with a high activation (CD107a+) of such cells. Nevertheless, no changes in infiltrations were seen with other immune populations such as CD8+ or CD4+ T cells among treated groups. Finally, a similar biodistribution was observed with around 1 μg/ml of Fc-fusion peptide in the tumor microenvironment (FIG. 14K) but below detection levels in the liver (FIG. 14L). In conclusion, Ad-Cab FT was able to control 4T1 tumor growth mostly due to the NK activation and downregulating MDSC populations.

### Ad-Cab FT is Effective in Controlling A549 Tumor Xenograft Model In Vivo

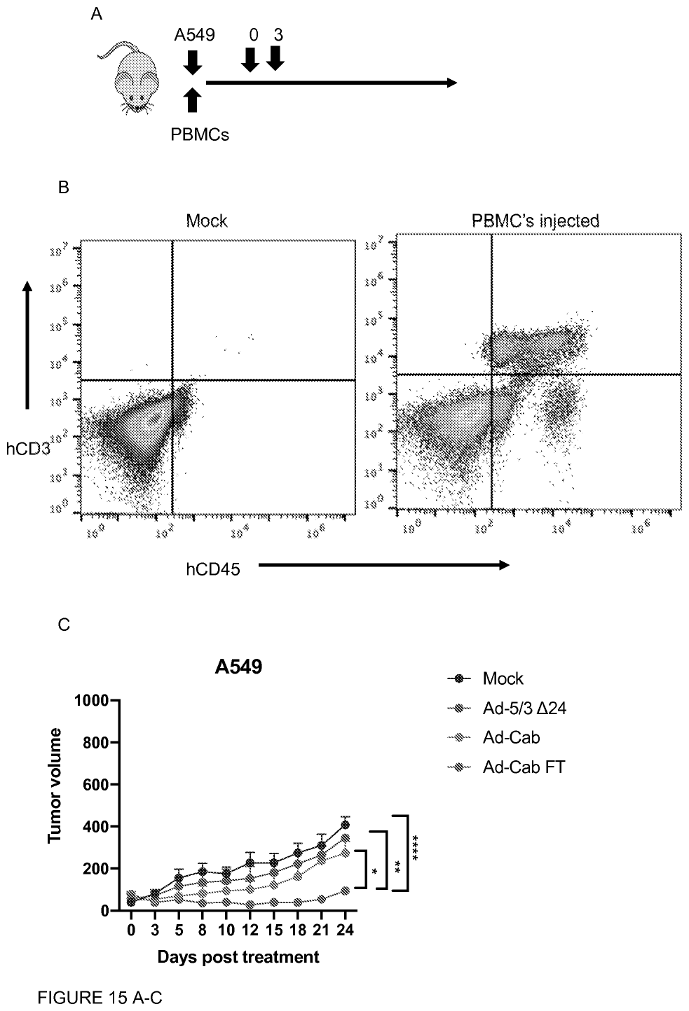

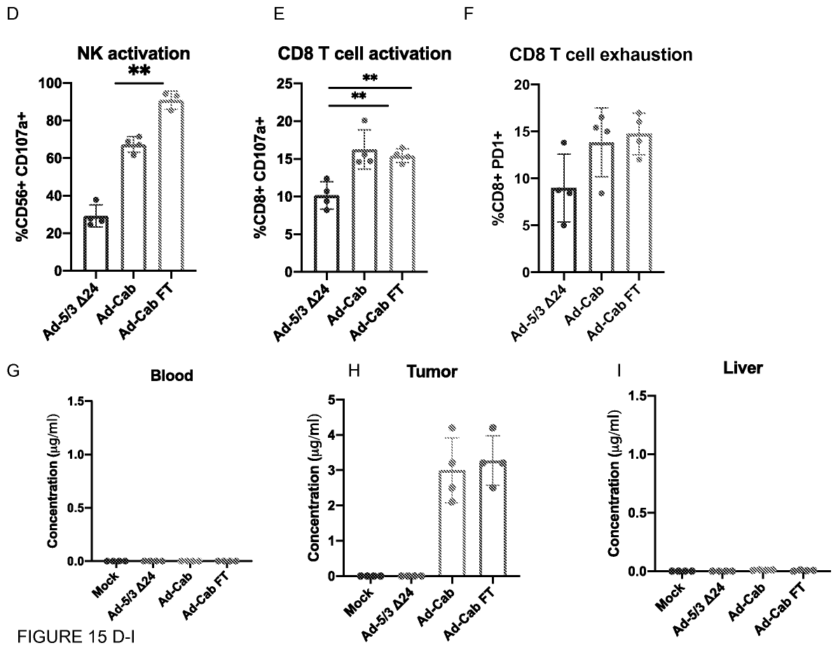

As a final tumor model to assess efficacy we used deficient NS (NOD/SCID) mice with a reconstituted human immune system and A549 tumor xenografts. NS mice were first implanted with A549 tumor cells and then injected with freshly isolated PBMCs from a healthy donor (FIG. 15A). Before treatment, two mice were sacrificed from mice injected with or without PBMCs. Mice injected with PBMCs could be seen to have engrafted human CD45+ cells and human CD3+ T cells (FIG. 15B). Mice were then treated with PBS (Mock), Ad-5/3 Δ24, Ad-Cab or Ad-Cab FT for a total of two injections. As expected, mice receiving Ad-Cab FT had the best tumor control compared to other groups (FIG. 15C). Ad-Cab did exert a therapeutic effect, yet it was mild and comparable to mice receiving Ad-5/3 Δ24. When examining the tumor microenvironment, both Ad-Cab and Ad-Cab FT mice groups had an upregulation of CD107a on NK cells indicating activation (FIG. 15D). Ad-Cab FT nevertheless had a higher upregulation of CD107a on NK cells compared to Ad-Cab which coincide with in vitro data. As for CD8 T cells, similar levels of CD107a (FIG. 15E) and PD1 (FIG. 15F) can be seen between Ad-Cab and Ad-Cab FT. These levels were higher compared to Ad-5/3 Δ24 group indicating an increase in T cell activation and exhaustion. Finally, we checked the bio-distribution of the Fc-fusion peptides. No Fc-fusion peptides were found in blood (FIG. 15G), yet a high amount could be observed in the tumor (FIG. 15H) and very minimal levels in the liver (FIG. 151). This indicated that most of the Fc-fusion peptide can be found in the tumor microenvironment with no leakage to the blood and liver. Thus, xenograft data further showed the effectiveness of Ad-Cab FT.

### CONCLUSIONS

At low concentrations, Ad-Cab FT was shown to secrete adequate levels of the Fc-fusion peptide able to induce higher tumor killing when PBMCs were added compared to Ad-Cab. Moreover, other than higher tumor, Ad-Cab FT was able to induce tumor cell death faster than Ad-Cab. The effectiveness of Ad-Cab FT was also seen in different in vivo models displaying better tumor control and higher activation of NK cells. Also, other than tumor control Ad-Cab FT was able to downregulate MDSC populations that have been correlated with poor prognosis and tumor growth. Finally, biodistribution analysis revealed that the oncolytic adenoviruses restricted the release of the toxic Fc-fusion peptides to the tumor circumventing safety concerns.

1. A cross-hybrid Fc-fusion polypeptide targeting PD-L1 (Programmed death-ligand 1), wherein the Fc-fusion polypeptide comprises an IgG and IgA Fc region and a region of PD-1 (Programmed cell death protein 1).

2. The Fc-fusion polypeptide of claim 1, wherein in the Fc-fusion polypeptide the IgG and IgA Fc region is connected to the region of PD-1 optionally via a linker, such as a glycine linker.

3. The Fc-fusion polypeptide of claim 1, wherein in the IgG and IgA Fc region IgG is IgG1 and/or IgA is IgA1.

4. The Fc-fusion polypeptide of claim 1, wherein the IgG and IgA Fc region comprises parts of the constant heavy chain (CH) 2 and/or 3 of an IgG and IgA;
parts of the CH2 of IgG1 and the CH3 of IgA1;
and/or part of the CH2 of IgG1, part of the CH2 of IgA1, and part of the CH3 of IgA1.

5. The Fc-fusion polypeptide of claim 1, wherein the IgG and IgA Fc region, such as IgG region, comprises one or more mutations and/or the glycosylation of the Fc region has been modified.

6. The Fc-fusion polypeptide of claim 5, wherein the mutation or mutations of the IgG region is/are selected from the group consisting of H268F, S324T, S239D and I332E.

7. The Fc-fusion polypeptide of claim 1, wherein the IgG and IgA Fc region is capable of binding one or more Fc-γ receptors and/or a Fc-α receptor.

8. The Fc-fusion polypeptide of claim 1, wherein the Fc-fusion polypeptide is capable of eliciting NK-mediated antibody-dependent cell cytotoxicity (ADCC); and/or
neutrophil-mediated ADCC, complement-dependent cytotoxicity (CDC) and/or antibody-dependent cell phagocytosis (ADCP).

9. The Fc-fusion polypeptide of claim 1, wherein the region of PD-1 comprises or is a PD-1 ectodomain.

10. The Fc-fusion polypeptide of claim 1, wherein the region of PD-1 or the region of PD-1 ectodomain comprises one or more mutations that optionally increase its affinity towards PD-L1 compared to a region of PD-1 or an ectodomain without said one or more mutations.

11. The Fc-fusion polypeptide of claim 1, wherein the Fc region comprises an amino acid sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 3 or 5;
the region of PD-1 comprises an amino acid sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 2; and/or a cross-hybrid Fc-fusion polypeptide targeting PD-L1 comprises an amino acid sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 1 or 4.

12. A polynucleotide encoding the Fc-fusion polypeptide of claim 1.

13. A vector, such as a viral vector, comprising the polynucleotide of claim 12.

14. The Fc-fusion polypeptide, of claim 1, wherein a polynucleotide encoding the Fc region comprises a polynucleotide sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 6;
a polynucleotide encoding the region of PD-1 comprises a polynucleotide sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 7; and/or
a polynucleotide encoding the cross-hybrid Fc-fusion polypeptide targeting PD-L1 comprises a polynucleotide sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 8 or 9.

15. The vector of claim 13, wherein
the vector is a viral vector, wherein a virus of the viral vector is a member of a family selected from the group consisting of Herpesviruses, Poxviruses, Hepadnaviruses, Flavivirus, Togavirus, Coronavirus, Hepatitis D, Orthomyxovirus, Paramyxovirus, Rhabdovirus, Bunyavirus, Filovirus and Retroviruses, and Adenovirus; and/or
the vector is an oncolytic viral vector or an oncolytic adenoviral vector selected from an Ad26, Chimp Ad, Gorilla Ad, Ad5, Ad3 or Ad5/3 vector.

16. A pharmaceutical composition comprising the Fc-fusion polypeptide of claim 1.

17. (canceled)

18. A method of treating a cancer in a subject, wherein the method comprises administering the Fc-fusion polypeptide of claim 1 to a subject in need thereof, wherein the cancer is selected from the group comprising nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer, and any combination thereof.

19. (canceled)

20. A method of preparing the cross-hybrid Fc-fusion polypeptide targeting PD-L1 of claim 1, wherein the method comprises allowing a polynucleotide encoding the cross-hybrid Fc-fusion polypeptide targeting PD-L1 to be expressed to said cross-hybrid Fc-fusion polypeptide in a cell.

21. (canceled)

22. The polynucleotide of claim 12, wherein the polynucleotide comprises:
a polynucleotide sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 6;
a polynucleotide encoding the region of PD-1 having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 7; and/or
a polynucleotide encoding the cross-hybrid Fc-fusion polypeptide targeting PD-L1 comprises a polynucleotide sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 8 or 9.

23. The vector of claim 13, wherein the vector comprises:
a polynucleotide encoding the Fc region and having a polynucleotide sequence having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 6;
a polynucleotide encoding the region of PD-1 and having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 7; and/or
a polynucleotide encoding the cross-hybrid Fc-fusion polypeptide targeting PD-L1 and having at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% sequence identity, or 100% sequence identity to SEQ ID NO: 8 or 9.

24. A pharmaceutical composition comprising the polynucleotide of claim 12.

25. A pharmaceutical composition comprising the vector of claim 13.

26. A method of treating a cancer in a subject, wherein the method comprises administering a polynucleotide of claim 12 to a subject in need thereof, wherein the cancer is selected from the group comprising nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer, and any combination thereof.

27. A method of treating a cancer in a subject, wherein the method comprises administering the vector of claim 13, to a subject in need thereof, wherein the cancer is selected from the group comprising nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer, and any combination thereof.


SEQUENCE LISTING (9 sequences)

SEQ ID NO: 1 - protein, 385 aa, Artificial Sequence
  Fc-fusion polypeptide
MEFGLSWVFLVALFRGVQCDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKAQIKESLRAELRVTERGGGGSGGGGSGGGGSPAPELLGGPSVFLFPPALEDLLLGSEANGVTCVVVDVSHEDPEVKFNWYVDGVEVHNAKTKPREEQYNSTYRVVSVLTVLHQDWLNGKEYKCKVSNKALPAPIEKTISKAKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRHHHHHHHH

SEQ ID NO: 2 - protein, 122 aa, Artificial Sequence
  PD-1 ectodomain
DSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKAQIKESLRAELRVTER

SEQ ID NO: 3 - protein, 221 aa, Artificial Sequence
  Fc of IgGA
PAPELLGGPSVFLFPPALEDLLLGSEANGVTCVVVDVSHEDPEVKFNWYVDGVEVHNAKTKPREEQYNSTYRVVSVLTVLHQDWLNGKEYKCKVSNKALPAPIEKTISKAKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDR

SEQ ID NO: 4 - protein, 385 aa, Artificial Sequence
  Modified (DF+TE) Fc-fusion polypeptide
MEFGLSWVFLVALFRGVQCDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKAQIKESLRAELRVTERGGGGSGGGGSGGGGSPAPELLGGPDVFLFPPALEDLLLGSEANGVTCVVVDVSFEDPEVKFNWYVDGVEVHNAKTKPREEQYNSTYRVVSVLTVLHQDWLNGKEYKCKVTNKALPAPEEKTISKAKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRHHHHHHHH

SEQ ID NO: 5 - protein, 221 aa, Artificial Sequence
  Modified (DF+TE) Fc of IgGA
PAPELLGGPDVFLFPPALEDLLLGSEANGVTCVVVDVSFEDPEVKFNWYVDGVEVHNAKTKPREEQYNSTYRVVSVLTVLHQDWLNGKEYKCKVTNKALPAPEEKTISKAKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDR

SEQ ID NO: 6 - DNA, 663 bp, Artificial Sequence
  Polynucleotide encoding the Fc region of the fusion polypeptide
cctgctccagaactgctcggcggaccttccgtgttcctgtttccaccagctctggaagatctgctgctgggctctgaagccaatggcgtgacatgtgtggtggtggacgtgtcccacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagagcggcaacaccttcagacccgaagtgcatctgctgcctccacctagcgaagaactggccctgaatgagctggtcaccctgacatgtctggccaggggcttcagccctaaggatgtgctcgttagatggctgcagggcagccaagagctgcccagagagaagtatctgacctgggccagcagacaagagcctagccagggcaccacaacctttgccgtgaccagcattctgagagtggccgccgaggattggaagaagggcgataccttcagctgcatggtcggacacgaagccctgcctctggccttcacacagaaaacaatcgaccgg

SEQ ID NO: 7 - DNA, 366 bp, Artificial Sequence
  Polynucleotide encoding the region of PD-1 of the fusion polypeptide
gacagccctgacagaccttggaaccctccaacattcagccccgctctgctggtggttaccgagggcgataatgccaccttcacctgtagcttcagcaacaccagcgagagcttccacgtcgtgtggcacagagagtctccaagcggccagacagataccctggctgcctttcctgaggacagatctcagcccggccaggactgcagattcagagtgacccagctgcctaacggccgggactttcacatgagcgtcgtgcgggccagaagaaacgatagcggcacatacgtgtgcggcgtgatctctctggcccctaaggctcagatcaaagagagcctgagagccgagctgagagtgaccgaaaga

SEQ ID NO: 8 - DNA, 1158 bp, Artificial Sequence
  Polynucleotide encoding the Fc-fusion polypeptide
atggaatttggcctgagctgggtgttcctggtggccctgtttagaggcgtgcagtgcgacagccctgacagaccttggaaccctccaacattcagccccgctctgctggtggttaccgagggcgataatgccaccttcacctgtagcttcagcaacaccagcgagagcttccacgtcgtgtggcacagagagtctccaagcggccagacagataccctggctgcctttcctgaggacagatctcagcccggccaggactgcagattcagagtgacccagctgcctaacggccgggactttcacatgagcgtcgtgcgggccagaagaaacgatagcggcacatacgtgtgcggcgtgatctctctggcccctaaggctcagatcaaagagagcctgagagccgagctgagagtgaccgaaagaggtggcggaggatctggcggaggtggaagcggcggaggcggatctgataagacccacacctgtcctccatgtcctgctccagaactgctcggcggaccttccgtgttcctgtttccaccagctctggaagatctgctgctgggctctgaagccaatggcgtgacatgtgtggtggtggacgtgtcccacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagagcggcaacaccttcagacccgaagtgcatctgctgcctccacctagcgaagaactggccctgaatgagctggtcaccctgacatgtctggccaggggcttcagccctaaggatgtgctcgttagatggctgcagggcagccaagagctgcccagagagaagtatctgacctgggccagcagacaagagcctagccagggcaccacaacctttgccgtgaccagcattctgagagtggccgccgaggattggaagaagggcgataccttcagctgcatggtcggacacgaagccctgcctctggccttcacacagaaaacaatcgaccgg

SEQ ID NO: 9 - DNA, 1158 bp, Artificial Sequence
  Polynucleotide encoding the modified (DF+TE) Fc-fusion polypeptide
atggaatttggcctgagctgggtgttcctggtggccctgtttagaggcgtgcagtgcgacagccctgacagaccttggaaccctccaacattcagccccgctctgctggtggttaccgagggcgataatgccaccttcacctgtagcttcagcaacaccagcgagagcttccacgtcgtgtggcacagagagtctccaagcggccagacagataccctggctgcctttcctgaggacagatctcagcccggccaggactgcagattcagagtgacccagctgcctaacggccgggactttcacatgagcgtcgtgcgggccagaagaaacgatagcggcacatacgtgtgcggcgtgatctctctggcccctaaggctcagatcaaagagagcctgagagccgagctgagagtgaccgaaagaggtggcggaggatctggcggaggtggaagcggcggaggcggatctgataagacccacacctgtcctccatgtcctgctccagaactgctcggcggacccgatgtgttcctgtttccaccagctctggaagatctgctgctgggctctgaagccaatggcgtgacatgtgtggtggtggacgtgtccttcgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaagtgaccaacaaggccctgcctgctcctgaggaaaagaccatcagcaaggccaagagcggcaacaccttcagacccgaagtgcatctgctgcctccacctagcgaagaactggccctgaatgagctggtcaccctgacatgtctggccaggggcttcagccctaaggatgtgctcgttagatggctgcagggcagccaagagctgcccagagagaagtatctgacctgggccagcagacaagagcctagccagggcaccacaacctttgccgtgaccagcattctgagagtggccgccgaggattggaagaagggcgataccttcagctgcatggtcggacacgaagccctgcctctggccttcacacagaaaaccatcgaccgg